(19) United States
(12) Patent Application Publication
(10) Pub. No.: US 2013/0045156 A1
(43) Pub. Date: Feb. 21, 2013

(54) METHOD FOR FORMING GRAPHENE AND CRAPHENE OXIDE SALT, AND GRAPHENE OXIDE SALT

(71) Applicants: Kuniharu Nomoto, Saitama (JP); Nobuhiro Inoue, Atsugi (JP); Mikio Yukawa, Atsugi (JP); Tatsuya Ikenuma, Atsugi (JP)

(72) Inventors: Kuniharu Nomoto, Saitama (JP); Nobuhiro Inoue, Atsugi (JP); Mikio Yukawa, Atsugi (JP); Tatsuya Ikenuma, Atsugi (JP)

(73) Assignee: SEMICONDUCTOR ENERGY LABORATORY CO., LTD., Atsugi-shi (JP)

(21) Appl. No.: 13/570,405

(22) Filed: Aug. 9, 2012

(30) Foreign Application Priority Data
Aug. 18, 2011 (JP) 2011-179215
Aug. 18, 2011 (JP) 2011-179217

(51) Int. Cl. C01B 31/02; C07C 51/16; C07C 51/29; C07C 51/305; C07C 233/64; C07C 63/42; C07C 39/235; C07C 231/10; C07C 37/64; B82Y 30/00; B82Y 40/00

(52) U.S. Cl. 423/445 R; 564/180; 562/408; 568/732; 977/847; 977/842; 977/734

(57) ABSTRACT

A first precipitate is formed by mixing graphite and an oxidizer containing an alkali metal salt in a solution. Next, a second precipitate is formed by ionizing the oxidizer which is included in the first precipitate, with an acid solution, and removing the oxidizer from the first precipitate. Then, a dispersion liquid in which graphene oxide is dispersed is prepared by mixing the second precipitate and water to form a mixed solution and then applying ultrasonic waves to the mixed solution or mechanically stirring the mixed solution, so that the graphene oxide is separated from graphite oxide that is the graphite which is included in the second precipitate and oxidized. Next, graphene oxide salt is formed by mixing the dispersion liquid, a basic solution, and an organic solvent and reacting the graphene oxide included in the dispersion liquid and a base included in the basic solution to each other.

### TECHNICAL FIELD

The present invention relates to a method for forming graphene and a method for forming graphene oxide salt, graphene oxide salt, and a power storage device and a semiconductor device each containing the graphene and the graphene oxide salt.

### BACKGROUND ART

In recent years, the use of graphene as a conductive electronic material in semiconductor devices has been studied. Graphene is a carbon layer in which six-membered rings each composed of carbon atoms are connected in the planar direction, and a stack of two or more and 100 or less carbon layers is referred to as multilayer graphene.

Graphene is chemically stable and has favorable electric characteristics; thus, its application to channel regions of transistors, vias, wirings, and the like included in semiconductor devices has been expected.

An active electrode material is coated with graphene in order that a material for an electrode of a lithium-ion battery can have higher conductivity.

As a method for forming graphene, graphite oxide or graphene oxide is reduced in the presence of a base. In order to form graphite oxide, a method in which sulfuric acid, nitric acid, and potassium chlorate are used as an oxidizer, a method in which sulfuric acid and potassium permanganate are used as an oxidizer, and a method in which potassium chlorate and fuming nitric acid are used as an oxidizer can be employed (see Patent Document 1).

### REFERENCE

### [Patent Document 1] Japanese Published Patent Application No. 2011-500488

### DISCLOSURE OF INVENTION

There is a Modified Hummers method as a method for forming graphene using graphite oxide which is obtained using graphite and, as an oxidizer, sulfuric acid and potassium permanganate. The method for forming graphene by a Modified Hummers method is described with reference to FIG. 3.

As illustrated in Step S101, graphite is oxidized by an oxidizer to form a mixed solution 1 containing graphite oxide. After that, in order to remove the remaining oxidizer, hydrogen peroxide and water are added to the mixed solution 1 and a mixed solution 2 is formed. Potassium permanganate which is not reacted yet is reduced by the hydrogen peroxide and then the reduced potassium permanganate is reacted with sulfuric acid, whereby manganese sulfate can be formed. Next, as illustrated in Step S102, the graphite oxide is collected from the mixed solution 2. Then, as illustrated in Step S103, the graphite oxide is cleaned using an acid solution in order to remove the remaining oxidizer. After that, the graphite oxide is diluted with a large amount of water and centrifugation is performed thereon, and then, as illustrated in Step S104, the graphite oxide, from which acid is separated, is collected. Next, as illustrated in Step S105, ultrasonic waves are applied to a mixed solution containing the collected graphite oxide and then an oxidized carbon layer in the graphite oxide is separated, so that graphene oxide is formed. After that, as illustrated in Step S106, reduction treatment for reducing oxygen bonded to the carbon layer is performed in an inert atmosphere to obtain graphene.

Although the step of cleaning the graphene oxide in Step S103 requires a large amount of water, it is possible to remove acid from the graphite oxide by repeating Step S103. However, when the acid content thereof becomes low, it is difficult to separate the graphite oxide that is a precipitate and acid included in a supernatant fluid; accordingly, the yield of the graphite oxide might be low. This results in low yield of graphene.

An electrode included in a power storage device includes a current collector and an active material layer. In the case of a conventional electrode, an active material layer includes a conductive additive, binder, and the like as well as an active material, and the discharge capacity per unit weight of the active material layer is decreased because of the conductive additive, the binder, and the like. Further, the binder included in the active material layer swells as it comes into contact with an electrolyte solution, so that the electrode is likely to be deformed and broken.

In view of the above problems, an object of one embodiment of the present invention is to form graphene and graphene oxide salt which is a raw material of graphene with high productivity. Another object of one embodiment of the present invention is to provide graphene oxide salt which is a raw material capable of forming graphene with high productivity. Another object of one embodiment of the present invention is to provide a power storage device having a higher discharge capacity and favorable electric characteristics. Another object of one embodiment of the present invention is to provide a power storage device having high reliability and high durability.

Graphene oxide salt according to one embodiment of the present invention is represented by the general formula (G1).

### [Chemical Formula 1]

Cₙ-A-B (G1)

(in the formula, n is a natural number, A represents any one of a carbonyl group, a carboxyl group, and a hydroxyl group, and B represents an ammonia group, an amino group, or an alkali metal.)

In other words, as a skeleton structure, the graphene oxide salt according to one embodiment of the present invention includes graphene represented by Cₙ in the above general formula; a carbonyl group, a carboxyl group, or a hydroxyl group represented by A in the above general formula, which is bonded to a carbon atom in graphene; and an ammonia group, an amino group, or an alkali metal represented by B in the above general formula, which is bonded to any one of the carbonyl group, the carboxyl group, and the hydroxyl group.

The graphene contains six-membered rings each composed of carbon atoms, which are connected in the planar direction, and poly-membered rings each formed when a carbon bond in part of a six-membered ring is broken, such as a seven-membered ring, an eight-membered ring, a nine-membered ring, and a ten-membered ring. A region surrounded with carbon atoms in the poly-membered ring becomes an opening.

When graphene oxide salt is heated in a reduced atmosphere or in a vacuum, the graphene oxide salt is reduced to be graphene. Therefore, it is possible to reduce graphene oxide salt and generate graphene by performing baking in a reduced atmosphere or in a vacuum.

One embodiment of the present invention is a method for forming graphene oxide salt through the following steps. First, a first precipitate is formed by mixing graphite and an oxidizer containing an alkali metal salt in a solution. Next, a second precipitate is formed by ionizing the oxidizer containing the alkali metal salt, which is included in the first precipitate, with an acid solution, and removing the oxidizer containing the alkali metal salt from the first precipitate. Then, a dispersion liquid in which graphene oxide is dispersed is prepared by mixing the second precipitate and water to form a mixed solution and then applying ultrasonic waves to the mixed solution or mechanically stirring the mixed solution, so that the graphene oxide is separated from graphite oxide that is the graphite which is included in the second precipitate and oxidized. Next, graphene oxide salt is formed by mixing the dispersion liquid, a basic solution, and an organic solvent and reacting the graphene oxide included in the dispersion liquid and a base included in the basic solution to each other.

Another embodiment of the present invention is a method for forming graphene oxide salt through the following steps. First, a first mixed solution containing a first precipitate having graphite oxide and an oxidizer is prepared by mixing graphite and the oxidizer in a solution. Next, a second precipitate containing the graphite oxide is formed by collecting the first precipitate from the first mixed solution and then removing the oxidizer from the first precipitate with an acid solution. Then, a second mixed solution in which graphene oxide is dispersed is prepared by mixing the second precipitate and water, and then applying ultrasonic waves to a mixed solution or mechanically stirring the mixed solution, so that the graphene oxide is separated from the graphite oxide. Next, graphene oxide salt is collected by mixing a basic solution and an organic solvent with the second mixed solution and reacting the graphene oxide included in the second mixed solution and a base to each other to precipitate the graphene oxide salt.

Another embodiment of the present invention is a method for forming graphene oxide salt through the following steps. First, a first precipitate is formed by mixing graphite and an oxidizer containing an alkali metal salt in a solution; and a second precipitate is formed by ionizing the oxidizer containing the alkali metal salt, which is included in the first precipitate, with an acid solution, and removing the oxidizer containing the alkali metal salt from the first precipitate. Next, a third precipitate containing graphite oxide salt is formed by mixing the second precipitate and water, then mixing a basic solution and an organic solvent, and reacting graphite oxide that is oxidized graphite, which is included in the second precipitate, and the basic solution. Then, graphene oxide is formed by mixing the third precipitate and water and separating graphene oxide salt from the graphite oxide salt included in the third precipitate.

Another embodiment of the present invention is a method for forming graphene oxide salt through the following steps. First, a first mixed solution containing a first precipitate having graphite oxide and an oxidizer is prepared by mixing graphite and the oxidizer in a solution. Next, a second precipitate containing the graphite oxide is formed by collecting the first precipitate from the first mixed solution and then removing the oxidizer from the first precipitate with an acid solution. Then, a third precipitate containing graphite oxide salt is formed by mixing the second precipitate and water, then mixing a basic solution and an organic solvent, and reacting the graphite oxide, which is included in the second precipitate, and the basic solution. Next, a second mixed solution in which graphene oxide salt is dispersed is prepared by mixing the third precipitate and water, and then applying ultrasonic waves to a mixed solution or mechanically stirring the mixed solution, so that the graphene oxide salt is separated from the graphite oxide salt included in the third precipitate; and the graphene oxide salt included in the second mixed solution is collected.

Another embodiment of the present invention is a method for forming graphene in a manner such that graphene is formed by reducing graphene oxide salt obtained by the above-described method.

Note that the oxidizer is nitric acid and potassium chlorate; sulfuric acid and potassium permanganate; or nitric acid, sulfuric acid, and potassium chlorate.

In addition, the acid solution is hydrochloric acid, dilute sulfuric acid, or nitric acid.

Further, the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

When a mixed solution containing graphite oxide, from which an oxidizer is removed, or graphene oxide is mixed with a basic solution and then with an organic solvent, graphite oxide salt or graphene oxide salt can be efficiently precipitated. Further, the graphene oxide salt is separated from the graphite oxide salt by applying ultrasonic waves to a mixed solution containing the graphite oxide salt or mechanically stirring the mixed solution. Reduction treatment is performed on the graphene oxide salt obtained by such methods, whereby graphene can be formed.

According to one embodiment of the present invention, graphene and graphene oxide salt which is a raw material of graphene can be formed with high productivity. In addition, graphene oxide salt which is a raw material of graphene can be provided. The use of graphene oxide salt for a positive electrode or a negative electrode of a power storage device makes it possible to increase discharge capacity of the power storage device. Further, the use of the graphene, instead of binder, for a positive electrode or a negative electrode can increase the reliability and durability of the power storage device.

### BRIEF DESCRIPTION OF DRAWINGS

FIG. 1 is a flow chart for illustrating a method for forming graphene oxide salt and graphene according to one embodiment of the present invention.

FIG. 2 is a flow chart for illustrating a method for forming graphene oxide salt and graphene according to one embodiment of the present invention.

FIG. 3 is a flow chart for illustrating a conventional method for forming graphene.

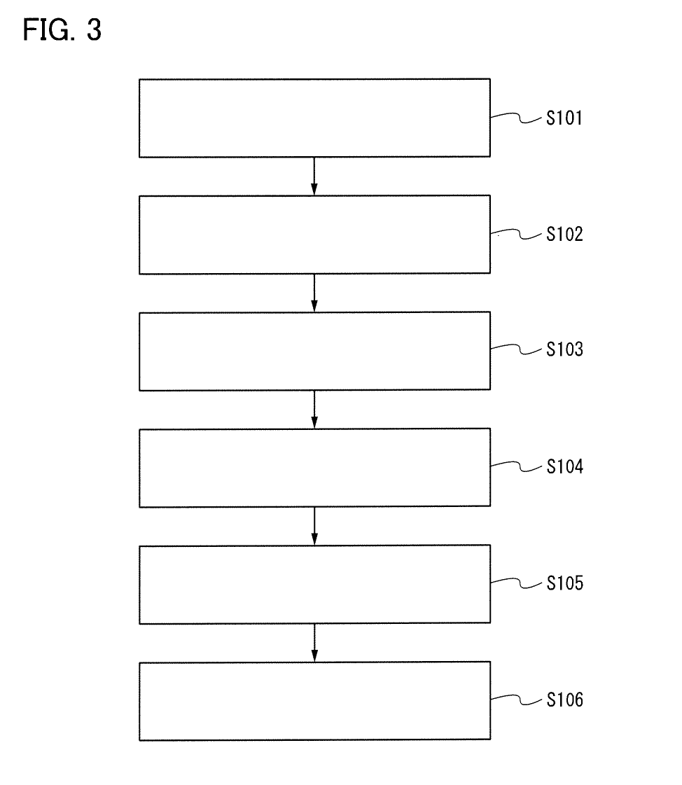

FIGS. 4A to 4D illustrate a negative electrode according to one embodiment of the present invention.

FIGS. 5A to 5C illustrate a positive electrode according to one embodiment of the present invention.

FIG. 6 illustrates a power storage device according to one embodiment of the present invention.

FIG. 7 illustrates electronic appliances.

FIG. 8 is a graph showing discharge characteristics and charge characteristics of a battery 1 and a comparative battery 1.

FIG. 9 is a graph showing ¹³C-NMR spectra.

FIG. 10 is a graph showing infrared absorption spectra.

### BEST MODE FOR CARRYING OUT THE INVENTION

Embodiments and examples of the present invention will be described below with reference to the accompanying drawings. Note that the embodiments and examples can be implemented in many different modes, and it is easily understood by those skilled in the art that various changes and modifications can be made without departing from the spirit and scope of the present invention. Therefore, the present invention should not be interpreted as being limited to the description in the following embodiments and examples.

### Embodiment 1

In this embodiment, graphene oxide salt according to one embodiment of the present invention will be described.

Graphene oxide salt which will be described in this embodiment is represented by the general formula (G1).

### [Chemical Formula 2]

Cₙ-A-B (G1)

(in the formula, n is a natural number, A represents any one of a carbonyl group, a carboxyl group, and a hydroxyl group, and B represents an ammonia group, an amino group, or an alkali metal.)

In other words, as a skeleton structure, the graphene oxide salt described in this embodiment includes graphene represented by Cₙin the above general formula; a carbonyl group, a carboxyl group, or a hydroxyl group represented by A in the above general formula, which is bonded to a carbon atom in graphene; and an ammonia group, an amino group, or an alkali metal represented by B in the above general formula, which is bonded to any one of the carbonyl group, the carboxyl group, and the hydroxyl group.

The graphene contains six-membered rings each composed of carbon atoms, which are connected in the planar direction, and poly-membered rings each formed when a carbon bond in part of a six-membered ring is broken, such as a seven-membered ring, an eight-membered ring, a nine-membered ring, and a ten-membered ring. A region surrounded with carbon atoms in the poly-membered ring becomes an opening.

Here, general formulae (G2) to (G9) which are specific examples of the general formula (G1) are shown below. Note that specific examples are not limited to the following general formulae (G2) to (G9).

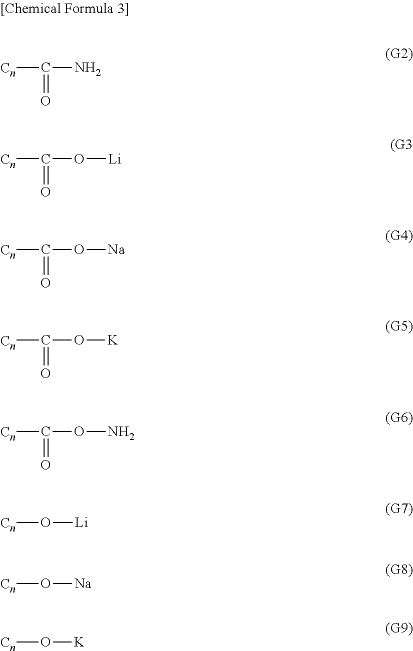

When graphene oxide salt is heated in a reduced atmosphere or in a vacuum, the graphene oxide salt is reduced to be graphene. Therefore, it is possible to form a positive or negative electrode active material layer and reduce the graphene oxide salt to be graphene by mixing a positive or negative electrode active material and the graphene oxide salt and by performing baking in a reduced atmosphere or in a vacuum.

### Embodiment 2

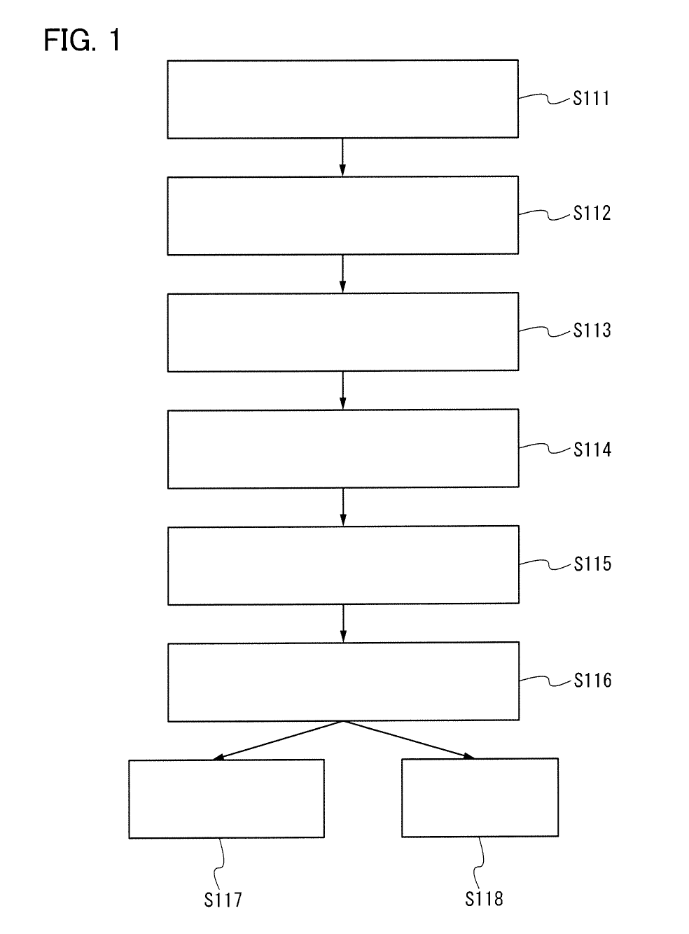

In this embodiment, a method for forming the graphene oxide salt, which is described in Embodiment 1, and graphene or multilayer graphene will be described with reference to FIG. 1.

FIG. 1 is a flow chart illustrating a process of forming graphene oxide salt, and graphene or multilayer graphene.

### <Oxidation Treatment of Graphite>

As illustrated in Step S111, graphite is oxidized by an oxidizer to form graphite oxide.

As an oxidizer, sulfuric acid, nitric acid and potassium chlorate; sulfuric acid and potassium permanganate; or potassium chlorate and fuming nitric acid are used. Here, graphite is oxidized by mixing graphite with sulfuric acid and potassium permanganate. Further, a mixed solution 1 containing the graphite oxide is formed by adding water.

After that, in order to remove the remaining oxidizer, hydrogen peroxide and water may be added to the mixed solution 1. Potassium permanganate which is not reacted yet is reduced by the hydrogen peroxide and then the reduced potassium permanganate is reacted with sulfuric acid, whereby manganese sulfate can be formed. Since the manganese sulfate is aqueous, it can be separated from the graphite oxide which is insoluble in water.

### <Collection of Graphite Oxide>

Next, as illustrated in Step S112, the graphite oxide is collected from the mixed solution 1. The mixed solution 1 is subjected to at least one of filtering, centrifugation, dialysis, and the like, so that a precipitate 1 containing the graphite oxide is collected from the mixed solution 1. Note that the precipitate 1 includes graphite which is not reacted yet.

### <Cleaning of Graphite Oxide>

Next, as illustrated in Step S113, a metal ion and a sulfate ion are removed from the precipitate 1 containing the graphite oxide with an acid solution. Here, a metal ion derived from the oxidizer, which is included in the precipitate 1 containing the graphite oxide, is dissolved in the acid solution, whereby a metal ion and a sulfate ion can be removed from the graphite oxide.

Graphite oxide contains a functional group such as a carbonyl group, a carboxyl group, or a hydroxyl group in an acid solution because oxygen is bonded to some carbon atoms in graphite. Therefore, the graphite oxide can be separated as a precipitate without dissolving in the acid solution. On the other hand, in a neutral solution or a basic solution, a functional group such as a carbonyl group, a carboxyl group, or a hydroxyl group, which is included in the graphite oxide, is easily ionized to be a carbonyl ion, a carboxyl ion, a hydroxide ion, or the like and is easily dissolved in a neutral solution or a basic solution. An acid solution is used to clean the graphite oxide because the yield of graphene which will be obtained later might be lowered if a neutral solution or a basic solution is used.

As typical examples of an acid solution, hydrochloric acid, dilute sulfuric acid, and nitric acid can be given. Note that the graphite oxide is preferably cleaned with acid having high volatility, typically hydrochloric acid, because the remaining acid solution is easily removed in a subsequent drying step.

As a method for removing a metal ion and a sulfate ion from the precipitate 1, there are a method in which the precipitate 1 and an acid solution are mixed and then a mixed solution is subjected to at least one of filtering, centrifugation, dialysis, and the like; a method in which the precipitate 1 is provided over a filter paper and then an acid solution is poured on the precipitate 1; and the like. Here, the precipitate 1 is provided over a filter paper, a metal ion and a sulfate ion are removed from the precipitate 1 by washing using the acid solution, and a precipitate 2 containing the graphite oxide is collected. Note that the precipitate 2 includes graphite which is not reacted yet.

### <Formation of Graphene Oxide>

Next, as illustrated in Step S114, the precipitate 2 is mixed with water and a mixed solution 2 in which the precipitate 2 is dispersed is prepared. Then, oxygen-contained carbon layers which are formed in the graphite oxide included in the mixed solution 2 are separated and graphene oxide is dispersed. Examples of a method for separating the graphene oxide from the graphite oxide are application of ultrasonic waves, mechanical stirring, and the like. Note that the mixed solution in which the graphene oxide is dispersed is to be a mixed solution 3.

The graphene oxide which is formed through this process contains six-membered rings each composed of carbon atoms, which are connected in the planar direction, and poly-membered rings each formed when a carbon bond in part of a six-membered ring is broken, such as a seven-membered ring, an eight-membered ring, a nine-membered ring, and a ten-membered ring. A region surrounded with carbon atoms in the poly-membered ring becomes an opening. In addition, a carbonyl group, a carboxyl group, or a hydroxyl group is bonded to one of the carbon atoms in the six-membered ring and the poly-membered ring. Note that instead of the dispersed graphene oxide, multilayer graphene oxide may be dispersed. The multilayer graphene oxide is composed of two or more and 100 or less carbon layers (graphene oxides) in each of which a carbonyl group, a carboxyl group, or a hydroxyl group is bonded to one of the carbon atoms in the six-membered ring and the poly-membered rings.

### <Collection of Graphene Oxide>

Next, as illustrated in Step S115, the mixed solution 3 is subjected to at least one of filtering, centrifugation, dialysis, and the like, whereby a mixed solution containing the graphene oxide and a precipitate 3 containing the graphite are separated from each other and the mixed solution containing the graphene oxide is collected. Note that the mixed solution containing the graphene oxide is to be a mixed solution 4.

In a mixed solution having polarity, such as water, different graphene oxides are not easily aggregated and thus dispersed because oxygen included in a carbonyl group, a carboxyl group, or a hydroxyl group is negatively charged.

### <Formation of Graphene Oxide Salt>

Next, as illustrated in Step S116, the mixed solution 4 is mixed with a basic solution to form graphene oxide salt. Then, an organic solvent is added to the graphene oxide salt and a mixed solution 5 in which the graphene oxide salt is precipitated as a precipitate 4 is prepared.

As typical examples of a basic solution, it is preferable to use a mixed solution which includes a base that reacts with the graphene oxide in neutralization without reducing oxygen bonded to a carbon atom of the graphene oxide, such as an aqueous sodium hydroxide solution, an aqueous pottasium hydroxide solution, an aqueous ammonia solution, a methylamine solution, an ethanolamine solution, a dimethylamine solution, or trimethylamine solution.

Since the organic solvent is used to precipitate the graphene oxide salt, acetone, methanol, ethanol, or the like is typically used as the organic solvent.

### <Collection of Graphene Oxide Salt>

Next, as illustrated in Step S117, the mixed solution 5 is subjected to at least one of filtering, centrifugation, dialysis, and the like, whereby the solvent and the precipitate 4 containing the graphene oxide salt are separated from each other and the precipitate 4 containing the graphene oxide salt is collected.

Next, the precipitate 4 is dried, so that the graphene oxide salt can be obtained.

In the graphene oxide salt, an ammonia group, an amino group, an alkali metal, or the like is bonded to a carbonyl group, a carboxyl group, or a hydroxyl group which is bonded to six-membered rings or poly-membered rings each composed of carbon atoms. Note that two or more and 100 or less layers of the graphene oxide salt may be stacked. Such stacked graphene oxide salt is referred to as multilayer graphene oxide salt.

### <Formation of Graphene>

Note that after Step S116, as illustrated in Step S118, graphene can be formed in a manner such that the mixed solution 5 containing the graphene oxide salt is provided over a substrate and then the graphene oxide salt is subjected to reduction treatment. Note that instead of the graphene, multilayer graphene might be formed in some cases.

As a method of providing the mixed solution containing the graphene oxide salt over the substrate, a coating method, a spin coating method, a dipping method, a spray method, an electrophoresis method, or the like may be employed. Alternatively, these methods may be combined as appropriate to be employed. For example, after the mixed solution containing the graphene oxide salt is applied to the substrate by a dipping method, the substrate is rotated as in a spin coating method, so that the evenness of the thickness of the mixed solution containing graphene oxide salt can be improved.

In the reduction treatment, heating is performed at higher than or equal to 150° C., preferably higher than or equal to 200° C. in a vacuum, in an inert gas (nitrogen, a rare gas, or the like) atmosphere, in the air, or the like. By being heated at a higher temperature and for a longer time, graphene oxide salt is easily reduced to a higher degree so that graphene with high purity (i.e., with a low concentration of elements other than carbon) can be obtained. Note that instead of the graphene, multilayer graphene might be formed in some cases.

Since graphite is treated with sulfuric acid according to the Modified Hummers method, a sulfone group and the like are also bonded to the graphite oxide, and its decomposition (release) turns out to start at around 300° C. Therefore, the graphene oxide salt is preferably reduced at 300° C. or higher.

Through the reduction treatment, adjacent graphenes are bonded to each other to form a huge net-like or sheet-like shape. Further, owing to the release of oxygen, an opening is formed in the graphene, which is a region surrounded with carbon atoms in a poly-membered ring, through the reduction treatment. Furthermore, the graphenes overlap with each other in parallel to a surface of the substrate. Thus, multilayer graphene is formed.

Note that oxygen remains in graphene or multilayer graphene which is obtained through the above formation method. As the proportion of oxygen becomes lower, the conductivity of the graphene or multilayer graphene can be higher. As the proportion of oxygen becomes higher, more openings serving as paths of ions can be formed in the graphene or multilayer graphene.

Through the above process, graphene oxide salt which is a raw material of graphene can be formed with high productivity. In addition, graphene or multilayer graphene can be formed with high productivity.

### Embodiment 3

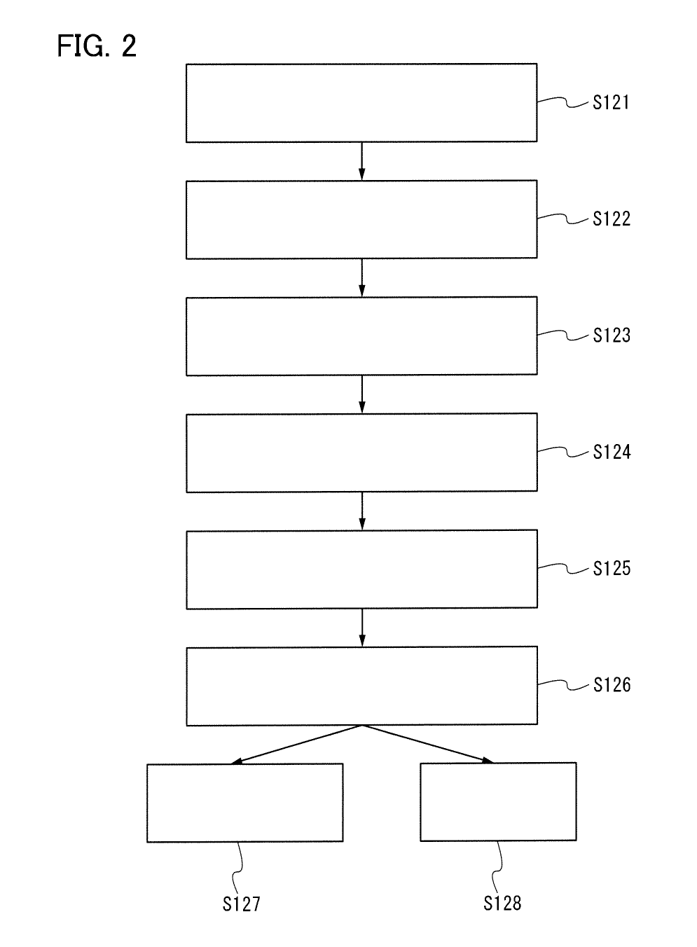

In this embodiment, a method for forming the graphene oxide salt which is described in Embodiment 1, and graphene or multilayer graphene by a method different from that of Embodiment 2 will be described with reference to FIG. 2. In this embodiment, after graphite oxide salt is formed, carbon layers in the graphite oxide salt are separated to form graphene oxide salt.

FIG. 2 is a flow chart illustrating a process of forming graphene oxide salt, and graphene or multilayer graphene.

### <Oxidation Treatment of Graphite>

As illustrated in Step S121, graphite is oxidized by an oxidizer to form graphite oxide. Further, a mixed solution 11 containing the graphite oxide is formed by adding water. Note that Step S121 may be performed in a manner similar to Step S111 described in Embodiment 2.

### <Collection of Graphite Oxide>

Next, as illustrated in Step S122, the graphite oxide is collected from the mixed solution 11. The mixed solution 11 is subjected to at least one of filtering, centrifugation, dialysis, and the like, so that a precipitate 11 containing the graphite oxide is collected from the mixed solution 11. Note that the precipitate 11 includes graphite which is not reacted yet. Note that Step S122 may be performed in a manner similar to Step S112 described in Embodiment 2.

### <Cleaning of Graphite Oxide>

Next, as illustrated in Step S123, a metal ion and a sulfate ion are removed from the precipitate 11 containing the graphite oxide with an acid solution. At this time, the precipitate from which a metal ion and a sulfate ion are removed is to be a precipitate 12. Note that the precipitate 12 includes graphite which is not reacted yet.

### <Formation of Graphite Oxide Salt>

Next, as illustrated in Step S124, after the precipitate 12 and water are mixed, a basic solution is mixed with a mixture to form graphite oxide salt. After that, an organic solvent is added to the graphite oxide salt, and a mixed solution 12 in which the graphite oxide salt that is a precipitate 13 is precipitated is prepared. As the basic solution and the organic solvent, those described in Embodiment 2 in Step S116 can be selected.

### <Collection of Graphite Oxide Salt>

Next, as illustrated in Step S125, the mixed solution 12 is subjected to at least one of filtering, centrifugation, dialysis, and the like, whereby the organic solvent and the precipitate 13 containing the graphite oxide salt are separated from each other and the precipitate 13 containing the graphite oxide salt is collected.

### <Formation of Graphene Oxide Salt>

Next, as illustrated in Step S126, the precipitate 13 is mixed with water and a mixed solution 13 in which the precipitate 13 is dispersed is formed. Then, oxygen-contained carbon layers which are formed in the graphite oxide salt included in the mixed solution 13 are separated and graphene oxide salt is dispersed. Examples of a method for separating the graphene oxide salt from the graphite oxide salt are application of ultrasonic waves, mechanical stirring, and the like. Note that the mixed solution in which the graphene oxide salt is dispersed is to be a mixed solution 14. Note that instead of the graphene oxide salt, multilayer graphene oxide salt might be formed in some cases.

### <Collection of Graphene Oxide Salt>

Next, as illustrated in Step S127, the mixed solution 14 is subjected to at least one of filtering, centrifugation, dialysis, and the like, whereby the precipitate 14 containing the graphene oxide salt is precipitated, and the precipitate 14 containing the graphene oxide salt is collected.

Next, the precipitate 14 is dried, so that the graphene oxide salt is obtained. Note that Step S127 may be performed in a manner similar to Step S117 described in Embodiment 2.

### <Formation of Graphene>

Note that after Step S126, as illustrated in Step S128, graphene or multilayer graphene can be formed in a manner such that the mixed solution 14 containing the graphene oxide salt is provided over a substrate and then the graphene oxide salt is subjected to reduction treatment.

The method of providing the mixed solution containing the graphene oxide salt over the substrate and the reduction treatment can be performed in a manner similar to those in Step S118 described in Embodiment 2.

Through the above process, graphene oxide salt which is a raw material of graphene can be formed with high productivity. In addition, graphene or multilayer graphene can be formed with high productivity.

### Embodiment 4

In this embodiment, a structure of an electrode of a power storage device and a formation method of the electrode will be described.

First, a negative electrode and a formation method thereof will be described.

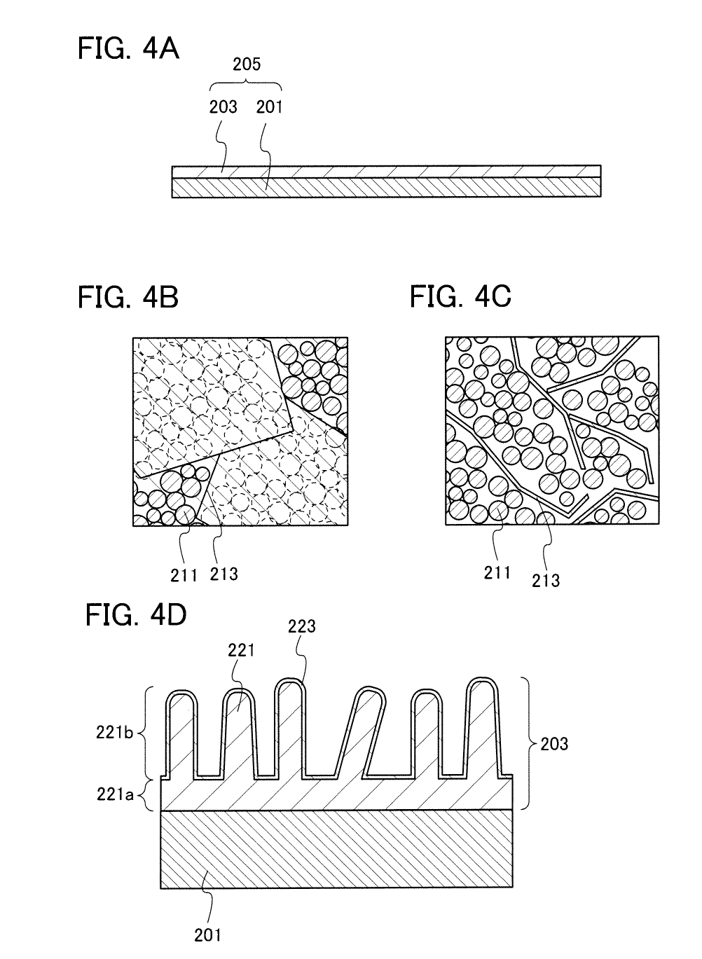

FIG. 4A is a cross-sectional view of a negative electrode 205. In the negative electrode 205, a negative electrode active material layer 203 is formed over a negative electrode current collector 201.

Note that the active material refers to a material that relates to injection and extraction of ions which function as carriers. Thus, the active material and the active material layer are distinguished.

As the negative electrode current collector 201, a material having high conductivity such as copper, stainless steel, iron, or nickel can be used. The negative electrode current collector 201 can have a foil shape, a plate shape, a net shape, or the like as appropriate.

The negative electrode active material layer 203 is formed using a negative electrode active material which can occlude and release ions serving as carriers. As typical examples of the negative electrode active material, lithium, aluminum, graphite, silicon, tin, and germanium are given. Further, a compound containing one or more of lithium, aluminum, graphite, silicon, tin, and germanium is given. Note that it is possible to omit the negative electrode current collector 201 and use the negative electrode active material layer 203 alone for a negative electrode. The theoretical capacity of germanium, silicon, lithium, and aluminum is higher that that of graphite as a negative electrode active material. When the occlusion capacity of a metal ion is high, the amount of negative electrode active material can be decreased, so that decreases in cost and size of a metal-ion secondary battery typified by a lithium-ion secondary battery can be achieved.

As examples of carrier ions used for metal-ion secondary batteries other than lithium-ion secondary batteries, alkali-metal ions such as sodium ions and potassium ions; alkaline-earth metal ions such as calcium ions, strontium ions, and barium ions; beryllium ions; and magnesium ions are given.

FIG. 4B is a plan view of the negative electrode active material layer 203. The negative electrode active material layer 203 contains a negative electrode active material 211 which is a particle capable of occluding and releasing carrier ions, and graphene or multilayer graphene 213 which covers a plurality of particles of the negative electrode active materials 211 and at least partly surrounds the plurality of particles of the negative electrode active materials 211. The different graphene or multilayer graphene 213 covers surfaces of the plurality of particles of the negative electrode active materials 211. The negative electrode active materials 211 may be partly exposed.

FIG. 4C is a cross-sectional view of part of the negative electrode active material layer 203 in FIG. 4B. The negative electrode active material layer 203 contains the negative electrode active materials 211 and the graphene or multilayer graphene 213 which at least partly surrounds the negative electrode active materials 211. The graphene or multilayer graphene 213 is observed to have linear shapes in cross section. A plurality of particles of the negative electrode active materials is at least partly surrounded with one graphene or plural graphenes. Alternatively, a plurality of particles of the negative electrode active materials is at least partly surrounded with one multilayer graphene or plural multilayer graphenes. Note that the graphene or multilayer graphene has a bag-like shape, and the plurality particles of the negative electrode active materials is at least partly surrounded with the bag-like portion in some cases. The graphene or multilayer graphene partly has openings where the negative electrode active materials are exposed in some cases.

The desired thickness of the negative electrode active material layer 203 is larger than or equal to 20 μm and smaller than or equal to 100 μm.

Note that the negative electrode active material layer 203 may contain acetylene black particles having a volume 0.1 to 10 times as large as that of the graphene or multilayer graphene, carbon particles having a one-dimensional expansion (e.g., carbon nanofibers), or other known binders.

The negative electrode active material layer 203 may be pre-doped with lithium. A lithium layer is formed on a surface of the negative electrode active material layer 203 by a sputtering method, whereby the negative electrode active material layer 203 can be pre-doped with lithium. Alternatively, lithium foil is provided on the surface of the negative electrode active material layer 203, whereby the negative electrode active material layer 203 can be pre-doped with lithium.

As an example of the negative electrode active material, a material whose volume is expanded by occlusion of ions serving as carriers can be given. When such a material is used, the negative electrode active material layer gets vulnerable and is partly collapsed by charging and discharging, resulting in lower reliability of a power storage device. However, the graphene or multilayer graphene 213 covering the periphery of the negative electrode active materials 211 allows prevention of dispersion of the negative electrode active materials and the collapse of the negative electrode active material layer, even when the volume of the negative electrode active materials is expanded due to charging and discharging. That is to say, the graphene or multilayer graphene has a function of maintaining the bond between the negative electrode active materials even when the volume of the negative electrode active materials fluctuates by charging and discharging.

The graphene or multilayer graphene 213 is in contact with a plurality of particles of the negative electrode active materials and serves also as an active material and a conductive additive. Further, the graphene or multilayer graphene 213 has a function of holding the negative electrode active materials capable of occluding and releasing carrier ions. Thus, a binder does not necessarily have to be mixed into the negative electrode active material layer. Accordingly, the amount of the negative electrode active materials in the negative electrode active material layer can be increased, which further allows an increase in discharge capacity of a power storage device because the graphene or multilayer graphene 213 also serves as an active material.

Next, a formation method of the negative electrode active material layer 203 in FIGS. 4B and 4C will be described.

Slurry containing negative electrode active materials which are particles and graphene oxide salt is formed. After the negative electrode current collector is coated with the slurry, heating is performed in a reduced atmosphere for reduction treatment so that the negative electrode active materials are baked and part of oxygen is released from the graphene oxide salt to form openings in the graphene or multilayer graphene, as in the formation method of graphene or multilayer graphene, which is described in Embodiment 2 or 3. Note that oxygen in the graphene oxide salt is not entirely reduced and part of oxygen remains in the graphene or multilayer graphene. Through the above process, the negative electrode active material layer 203 can be formed over the negative electrode current collector 201.

Next, a structure of a negative electrode in FIG. 4D will be described.

FIG. 4D is a cross-sectional view of the negative electrode where the negative electrode active material layer 203 is formed over the negative electrode current collector 201. The negative electrode active material layer 203 contains a negative electrode active material 221 having an uneven surface and graphene or multilayer graphene 223 covering a surface of the negative electrode active material 221.

The uneven negative electrode active material 221 includes a common portion 221a and a projected portion 221b extending from the common portion 221a. The projected portion 221b can have a columnar shape such as a cylinder shape or a prism shape, or a needle shape such as a cone shape or a pyramid shape, as appropriate. The top of the projected portion may be curved. The negative electrode active material 221 is formed using a negative electrode active material capable of occluding and releasing ions serving as carriers, typically, lithium ions, in a manner similar to that of the negative electrode active material 211. Note that the common portion 221 a and the projected portion 221b may be formed using the same material. Alternatively, the common portion 221a and the projected portion 221b may be formed using different materials.

In the case of silicon which is an example of a negative electrode active material, the volume is approximately quadrupled due to occlusion of ions serving as carriers; therefore, the negative electrode active material 221 gets vulnerable and part of the negative electrode active material layer 203 is collapsed by charging and discharging, resulting in lower reliability of a power storage device. However, the graphene or multilayer graphene 223 covering the periphery of the negative electrode active material 221 allows suppression of dispersion of the negative electrode active material and the collapse of the negative electrode active material layer 203, even when the volume of silicon is expanded due to charging and discharging; accordingly, reliability and durability of a power storage device can be increased.

When the surface of the negative electrode active material layer 203 is in contact with an electrolyte, the electrolyte and the negative electrode active material react with each other, so that a film is formed over a surface of the negative electrode. The film is called a solid electrolyte interface (SEI) and considered necessary to relieve the reaction of the electrode and the electrolyte for stabilization. However, when the thickness of the film is increased, carrier ions are less likely to be occluded in the negative electrode, leading to problems such as a reduction in conductivity of carrier ions between the electrode and the electrolyte solution, a decrease in discharge capacity due to the reduction in conductivity thereof, and a waste of the electrolyte solution.

The graphene or multilayer graphene coating the surface of the negative electrode active material layer 203 can suppress an increase in thickness of the film, so that a decrease in discharge capacity can be suppressed.

Next, a formation method of the negative electrode active material layer 203 in FIG. 4D will be described.

An uneven negative electrode active material is provided over a negative electrode current collector by a printing method, an ink-jet method, a CVD method, or the like. Alternatively, a negative electrode active material having a film shape is formed by a coating method, a sputtering method, an evaporation method, or the like, and then is selectively removed, so that the uneven negative electrode active material is provided over a negative electrode current collector. Still alternatively, a surface of foil or a plate which is formed of lithium, aluminum, graphite, or silicon is partly removed to form a negative electrode current collector and a negative electrode active material that have an uneven shape. Further alternatively, a net formed of lithium, aluminum, graphite, or silicon may be used for a negative electrode and a negative electrode current collector.

After that, the mixed solution containing the graphene oxide salt is provided over the negative electrode active material as in Embodiment 2. As a method of providing the mixed solution containing the graphene oxide salt over the negative electrode active material, a coating method, a spin coating method, a dipping method, a spray method, an electrophoresis method, or the like may be employed. Then, heating is performed in a reduced atmosphere for reduction treatment so that part of oxygen is released from the graphene oxide salt provided in the negative active material to form openings in the graphene or multilayer graphene, as in the formation method of graphene or multilayer graphene which is described in Embodiment 2. Note that oxygen in the graphene oxide salt is not entirely released and oxygen that is not released remains in the graphene or multilayer graphene. Through the above process, the negative electrode active material layer 203 coated with the graphene or multilayer graphene 223 can be formed over a surface of the negative electrode active material 221.

The use of the mixed solution containing the graphene oxide salt in formation of the graphene or multilayer graphene permits a surface of the uneven negative electrode active material to be coated with the graphene or multilayer graphene having an even thickness.

Note that the uneven negative electrode active material (hereinafter referred to as a silicon whisker) formed of silicon can be provided over the negative electrode current collector by an LPCVD method using silane, silane chrolide, silane fluoride, or the like as a source gas.

The silicon whiskers may be amorphous. When an amorphous silicon whisker is used for the negative electrode active material layer, the volume is less likely to be changed due to occlusion and release of ions serving as carriers (e.g., stress caused by expansion in volume is relieved). For this reason, the negative electrode active material layer can be prevented from being turned into fine powder and separated by repeated cycles of charge and discharge; accordingly, a power storage device whose cycle characteristics are further improved can be fabricated.

Alternatively, the silicon whisker may be crystalline. In this case, the crystalline region which has excellent conductivity and ion mobility is in contact with the negative electrode current collector in a wide range of area. Therefore, it is possible to further improve the conductivity of the entire negative electrode, which enables charging and discharge to be performed at much higher speed; accordingly, a power storage device whose charge-discharge capacity is improved can be fabricated.

Further alternatively, the silicon whisker may include a core, which is a crystalline region, and an outer shell covering the core, which is an amorphous region.

The amorphous outer shell has a characteristic that the volume is less likely to be changed due to occlusion and release of ions (e.g., stress caused by expansion in volume is relieved). In addition, the crystalline core, which has excellent conductivity and ion mobility, has a characteristic that the rate of occluding ions and the rate of releasing ions are high per unit mass. Thus, when the silicon whisker having the core and the outer shell is used for the negative electrode active material layer, charging and discharging can be performed at high speed; accordingly, a power storage device whose charge-discharge capacity and cycle characteristics are improved can be fabricated.

In the case of silicon which is an example of a negative electrode active material, the volume is approximately quadrupled due to occlusion of ions serving as carriers. When such a material is used, the negative electrode active material layer gets vulnerable and is partly collapsed by charging and discharging, resulting in lower reliability of a power storage device. However, the graphene or multilayer graphene covering surfaces of the silicon whisker allows suppression of the collapse of the negative electrode active material layer due to expansion of the volume of the silicon whisker; accordingly, reliability and durability of a power storage device can be increased.

Next, a positive electrode and a formation method thereof will be described.

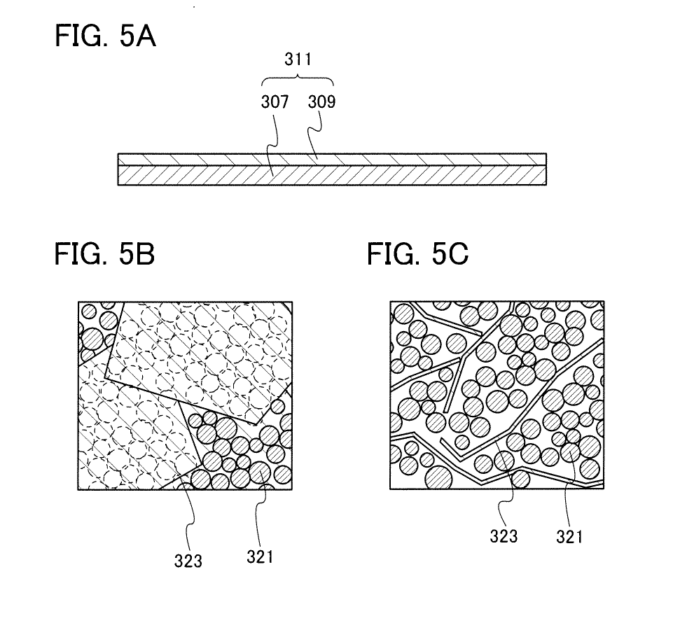

FIG. 5A is a cross-sectional view of a positive electrode 311. In the positive electrode 311, a positive electrode active material layer 309 is formed over a positive electrode current collector 307.

As the positive electrode current collector 307, a material having high conductivity such as platinum, aluminum, copper, titanium, or stainless steel can be used. The positive electrode current collector 307 can have a foil shape, a plate shape, a net shape, or the like as appropriate.

The positive electrode active material layer 309 can be formed using LiFeO₂, LiCoO₂, LiNiO₂, LiMn₂O₄, V₂O₅, Cr₂O₅, MnO₂, or the like as a material.

Alternatively, an olivine-type lithium-containing composite oxide (a general formula LiMPO₄ (M is one or more of Fe(II), Mn(II), Co(II), and Ni(II))) can be used. Typical examples of the general formula LiMPO₄ which can be used as a material are lithium compounds such as LiFePO₄, LiNiPO₄, LiCoPO₄, LiMnPO₄, LiFeₐNi_{b}PO₄, LiFeₐCo_{b}PO₄, LiFeₐMn_{b}PO₄, LiNiₐCo_{b}PO₄, LiNiₐMn_{b}PO₄ (a+b≦1, 0<a<1, and 0<b <1), LiFe_{c}Ni_{d}CoₑPO₄, LiFe_{c}Ni_{d}MnₑPO₄, LiNi_{c}Co_{d}MnₑPO₄ (c+d+e≦1, 0<e<1, 0<d <1, and 0<e<1), and LiFeᵣNi_{g}CoₕMnᵢPO₄ (f+g+h+i≦1, 0<f<1, 0<g<1, 0<h<1, and 0<i<1).

Alternatively, a lithium-containing composite oxide such as a general formula Li₂MSiO₄ (M is one or more of Fe(II), Mn(II), Co(II), and Ni(II)) can be used. Typical examples of the general formula Li₂MSiO₄ which can be used as a material are lithium compounds such as Li₂FeSiO₄, Li₂NiSiO₄, Li₂CoSiO₄, Li₂MnSiO₄, Li₂FeₖNiₗSiO₄, Li₂FeₖCo/SiO₄, Li₂FeₖMnᵢSiO₄, Li₂NiₖCo/SiO₄, Li₂NiₖMnᵢSiO₄ (k+1≦1,0<k<1, and 0<l<1), Li₂FeₘNiₙCo_{g}SiO₄, Li₂FeₘNiₙMn_{q}SiO₄, Li₂NiₘCoₙMn_{q}SiO₄ (m+n+q≦1, 0<m<1, 0<n<1, and 0<q<1), and Li₂FeᵣNiₛCo_{f}MnᵤSiO₄ (r+s+t+u23 1, 0<r<1, 0<s<1, 0<t<1, and 0<u<1).

In the case where carrier ions are alkali metal ions other than lithium ions, alkaline-earth metal ions, beryllium ions, or magnesium ions, the positive electrode active material layer 309 may contain, instead of lithium in the lithium compound and the lithium-containing composite oxide, an alkali metal (e.g., sodium or potassium), an alkaline-earth metal (e.g., calcium, strontium, or barium), beryllium, or magnesium.

FIG. 5B is a plan view of the positive electrode active material layer 309. The positive electrode active material layer 309 contains a positive electrode active material 321 which is a particle capable of occluding and releasing carrier ions, and graphene or multilayer graphene 323 which covers a plurality of particles of the positive electrode active materials 321 and at least partly surrounds the plurality of particles of the positive electrode active materials 321. The different graphene or multilayer graphene 323 covers surfaces of the plurality of particles of the positive electrode active materials 321. The positive electrode active materials 321 may partly be exposed.

The size of the particle of the positive electrode active material 321 is preferably greater than or equal to 20 nm and less than or equal to 100 nm. Note that the size of the particle of the positive electrode active material 321 is preferably smaller because electrons transfer in the positive electrode active materials 321.

In the case where the positive electrode active material layer 309 contains the graphene or multilayer graphene 323, sufficient characteristics can be obtained even when surfaces of the positive electrode active materials 321 are not coated with a carbon film; however, it is preferable to use both the graphene or multilayer graphene 323 and the positive electrode active material coated with a carbon film because electrons transfers hopping between the positive electrode active materials.

FIG. 5C is a cross-sectional view of part of the positive electrode active material layer 309 in FIG. 5B. The positive electrode active material layer 309 contains the positive electrode active materials 321 and the graphene or multilayer graphene 323 which covers the positive electrode active materials 321. The graphene or multilayer graphene 323 is observed to have linear shapes in cross section. A plurality of particles of the positive electrode active materials is at least partly surrounded with one graphene or plural graphenes. Alternatively, a plurality of particles of the positive electrode active materials is at least partly surrounded with one multilayer graphene or plural multilayer graphenes. Note that the graphene or multilayer graphene has a bag-like shape, and the plurality particles of the positive electrode active materials is at least partly surrounded with the bag-like portion in some cases. The graphene or multilayer graphene partly has openings where the positive electrode active materials are exposed in some cases.

The desired thickness of the positive electrode active material layer 309 is larger than or equal to 20 μm and smaller than or equal to 100 μm. It is preferable to adjust the thickness of the positive electrode active material layer 309 as appropriate so that a crack and separation are not caused.

Note that the positive electrode active material layer 309 may contain acetylene black particles having a volume 0.1 to 10 times as large as that of the graphene or multilayer graphene, carbon particles having a one-dimensional expansion (e.g., carbon nanofibers), or other known binders.

As an example of the positive electrode active material, a material whose volume is expanded by occlusion of ions serving as carriers can be given. When such a material is used, the positive electrode active material layer gets vulnerable and is partly collapsed by charging and discharging, resulting in lower reliability of a power storage device. However, the graphene or multilayer graphene 323 covering the periphery of the positive electrode active materials allows prevention of dispersion of the positive electrode active materials and the collapse of the positive electrode active material layer, even when the volume of the positive electrode active materials is expanded due to charging and discharging. That is to say, the graphene or multilayer graphene has a function of maintaining the bond between the positive electrode active materials even when the volume of the positive electrode active materials fluctuates by charging and discharging.

The graphene or multilayer graphene 323 is in contact with a plurality of particles of the positive electrode active materials and serves also as a conductive additive. Further, the graphene or multilayer graphene 323 has a function of holding the positive electrode active materials 321 capable of occluding and releasing carrier ions. Thus, binder does not necessarily have to be mixed into the positive electrode active material layer. Accordingly, the amount of the positive electrode active materials in the positive electrode active material layer can be increased, which allows an increase in discharge capacity of a power storage device,

Next, a formation method of the positive electrode active material layer 309 will be described.

Slurry containing positive electrode active materials which are particles and graphene oxide salt is formed. After the positive electrode current collector is coated with the slurry, heating is performed in a reduced atmosphere for reduction treatment so that the positive electrode active materials are baked and oxygen included in the graphene oxide salt is released to form openings in the graphene or multilayer graphene 323, as in the formation method of graphene or multilayer graphene, which is described in Embodiment 2. Note that oxygen in the graphene oxide salt is not entirely reduced and part of oxygen remains in the graphene or multilayer graphene 323. Through the above process, the positive electrode active material layer 309 can be formed over the positive electrode current collector 307. Consequently, the positive electrode active material layer has higher conductivity.

Oxygen included in the graphene oxide salt is negatively charged in a polar solvent. As a result of being negatively charged, graphene oxide salt is dispersed. Accordingly, the positive electrode active materials included in the slurry are not easily aggregated, so that the size of the particle of the positive electrode active material can be prevented from increasing due to backing. Thus, the transfer of electrons in the positive electrode active materials is facilitated, resulting in an increase in conductivity of the positive electrode active material layer.

### Embodiment 5

In this embodiment, a method for fabricating a power storage device will be described.

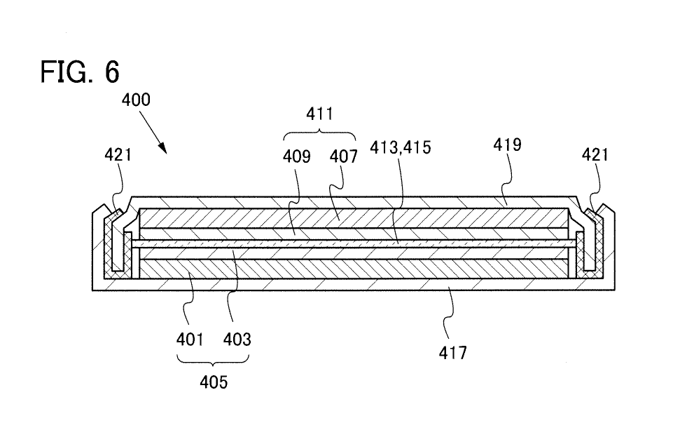

A lithium-ion secondary battery which is a typical example of power storage devices of this embodiment will be described with reference to FIG. 6. Here, description is made below on a cross-sectional structure of the lithium-ion secondary battery.

FIG. 6 is a cross-sectional view of the lithium-ion secondary battery.

A lithium-ion secondary battery 400 includes a negative electrode 411 including a negative electrode current collector 407 and a negative electrode active material layer 409, a positive electrode 405 including a positive electrode current collector 401 and a positive electrode active material layer 403, and a separator 413 provided between the negative electrode 411 and the positive electrode 405. Note that the separator 413 includes an electrolyte 415. The negative electrode current collector 407 is connected to an external terminal 419 and the positive electrode current collector 401 is connected to an external terminal 417. An end portion of the external terminal 419 is embedded in a gasket 421. In other words, the external terminals 417 and 419 are insulated from each other by the gasket 421.

As the negative electrode current collector 407 and the negative electrode active material layer 409, the negative electrode current collector 201 and the negative electrode active material layer 203 which are described in Embodiment 3 can be used as appropriate.

As the positive electrode current collector 401 and the positive electrode active material layer 403, the positive electrode current collector 307 and the positive electrode active material layer 309 which are described in Embodiment 3 can be used as appropriate.

An insulating porous material is used for the separator 413. Typical examples of the separator 413 include cellulose (paper), polyethylene, and polypropylene.

As a solute of an electrolyte 415, a material in which carrier ions can transfer and exist stably is used. Typical examples of the solute of the electrolyte include lithium salt such as LiClO₄, LiAsF₆, LiBF₄, LiPF₆, and Li(C₂F₅SO₂)₂N.

In the case where carrier ions are alkali metal ions other than lithium ions, alkaline-earth metal ions, beryllium ions, or magnesium ions, the solute of the electrolyte 415 may contain, instead of lithium in the lithium salt, an alkali metal (e.g., sodium or potassium), an alkaline-earth metal (e.g., calcium, strontium, or barium), beryllium, or magnesium.

As a solvent of the electrolyte 415, a material in which lithium ions can transfer is used. As the solvent of the electrolyte 415, an aprotic organic solvent is preferably used. Typical examples of aprotic organic solvents include ethylene carbonate, propylene carbonate, dimethyl carbonate, diethyl carbonate, γ-butyrolactone, acetonitrile, dimethoxyethane, and tetrahydrofuran, and one or more of these materials can be used. When a gelled polymer is used as the solvent of the electrolyte 415, safety against liquid leakage or the like is increased. Further, the lithium-ion secondary battery 400 can be made thinner and more lightweight. Typical examples of gelled polymers include a silicon gel, an acrylic gel, an acrylonitrile gel, polyethylene oxide, polypropylene oxide, and a fluorine-based polymer.

As the electrolyte 415, a solid electrolyte such as Li₃PO₄ can be used. Note that in the case where the solid electrolyte is used as the electrolyte 415, the separator 413 is unnecessary.

For the external terminals 417 and 419, a metal member such as a stainless steel plate or an aluminum plate can be used as appropriate.

Note that in this embodiment, a coin-type lithium-ion secondary battery is given as the lithium-ion secondary battery 400; however, any of lithium-ion secondary batteries with various shapes, such as a sealing-type lithium-ion secondary battery, a cylindrical lithium-ion secondary battery, and a square-type lithium-ion secondary battery, can be used. Further, a structure in which a plurality of positive electrodes, a plurality of negative electrodes, and a plurality of separators are stacked or rolled may be employed.

A lithium-ion secondary battery has a high energy density, a large capacity, and further a high output voltage. Thus, small size and low weight of the lithium-ion secondary battery can be achieved. Further, the lithium-ion secondary battery is not easily degraded due to repetitive charge and discharge and can be used for a long time, so that cost can be decreased.

Next, a method for fabricating the lithium-ion secondary battery 400 described in this embodiment will be described.

By the fabricating method described in Embodiment 4, the positive electrode 405 and the negative electrode 411 are formed.

Next, the positive electrode 405, the separator 413, and the negative electrode 411, are impregnated with the electrolyte 415. Then, the positive electrode 405, the separator 413, the gasket 421, the negative electrode 411, and the external terminal 419 are stacked in this order over the external terminal 417, and the external terminal 417 and the external terminal 419 are crimped to each other with a “coin cell crimper”. Thus, the coin-type lithium-ion secondary battery can be fabricated.

Note that a spacer and a washer may be provided between the external terminal 417 and the positive electrode 405 or between the external terminal 419 and the negative electrode 411 so that connection between the external terminal 417 and the positive electrode 405 or between the external terminal 419 and the negative electrode 411 is enhanced.

### Embodiment 6

The power storage device according to one embodiment of the present invention can be used for power supplies of a variety of electronic appliances which can be operated with power.

Specific examples of electronic appliances using the power storage device according to one embodiment of the present invention are as follows: display devices, lighting devices, desktop personal computers or laptop personal computers, image reproduction devices which reproduce a still image or a moving image stored in a recording medium such as a digital versatile disc (DVD), mobile phones, portable game machines, portable information terminals, e-book readers, cameras such as video cameras and digital still cameras, high-frequency heating apparatus such as microwaves, electric rice cookers, electric washing machines, air-conditioning systems such as air conditioners, electric refrigerators, electric freezers, electric refrigerator-freezers, freezers for preserving DNA, and dialysis devices. In addition, moving objects driven by an electric motor using power from a power storage device are also included in the category of electronic appliances. As examples of the moving objects, electric vehicles, hybrid vehicles which include both an internal-combustion engine and a motor, and motorized bicycles including motor-assisted bicycles can be given.

In the electronic appliances, the power storage device according to one embodiment of the present invention can be used as a power storage device for supplying enough power for almost the whole power consumption (such a power storage device is referred to as a main power supply). Alternatively, in the electronic appliances, the power storage device according to one embodiment of the present invention can be used as a power storage device which can supply power to the electronic appliances when the supply of power from the main power supply or a commercial power supply is stopped (such a power storage device is referred to as an uninterruptible power supply). Further alternatively, in the electronic appliances, the power storage device according to one embodiment of the present invention can be used as a power storage device for supplying power to the electronic appliances at the same time as the power supply from the main power supply or a commercial power supply (such a power storage device is referred to as an auxiliary power supply).

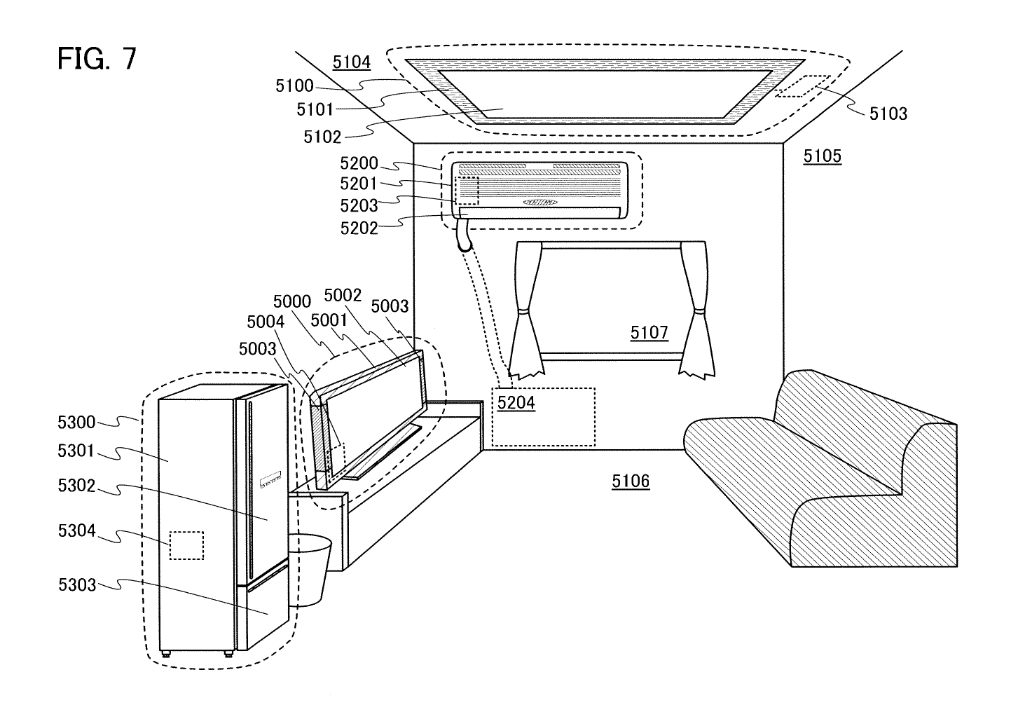

FIG. 7 illustrates specific structures of the electronic appliances. In FIG. 7, a display device 5000 is an example of an electronic appliance including a power storage device 5004 according to one embodiment of the present invention. Specifically, the display device 5000 corresponds to a display device for TV broadcast reception and includes a housing 5001, a display portion 5002, speaker portions 5003, the power storage device 5004, and the like. The power storage device 5004 according to one embodiment of the present invention is provided inside the housing 5001. The display device 5000 can receive power from a commercial power supply. Alternatively, the display device 5000 can use power stored in the power storage device 5004. Thus, the display device 5000 can be operated with the use of the power storage device 5004 according to one embodiment of the present invention as an uninterruptible power supply even when power cannot be supplied from the commercial power supply due to power failure or the like.

A semiconductor display device such as a liquid crystal display device, a light-emitting device in which a light-emitting element such as an organic EL element is provided in each pixel, an electrophoretic display device, a digital micromirror device (DMD), a plasma display panel (PDP), and a field emission display (FED) can be used for the display portion 5002.

Note that the display device includes, in its category, all of information display devices for personal computers, advertisement displays, and the like other than TV broadcast reception.

In FIG. 7, an installation lighting device 5100 is an example of an electronic appliance including a power storage device 5103 according to one embodiment of the present invention. Specifically, the installation lighting device 5100 includes a housing 5101, a light source 5102, the power storage device 5103, and the like. Although FIG. 7 illustrates the case where the power storage device 5103 is provided in a ceiling 5104 on which the housing 5101 and the light source 5102 are installed, the power storage device 5103 may be provided in the housing 5101. The installation lighting device 5100 can receive power from the commercial power supply. Alternatively, the installation lighting device 5100 can use power stored in the power storage device 5103. Thus, the installation lighting device 5100 can be operated with the use of the power storage device 5103 according to one embodiment of the present invention as an uninterruptible power supply even when power cannot be supplied from the commercial power supply due to power failure or the like.

Note that although the installation lighting device 5100 provided in the ceiling 5104 is illustrated in FIG. 7 as an example, the power storage device according to one embodiment of the present invention can be used in an installation lighting device provided in, for example, a wall 5105, a floor 5106, a window 5107, or the like other than the ceiling 5104. Alternatively, the power storage device can be used in a tabletop lighting device and the like.

As the light source 5102, an artificial light source which provides light artificially by using power can be used. Specifically, a discharge lamp such as an incandescent lamp and a fluorescent lamp, and a light-emitting element such as an LED and an organic EL element are given as examples of the artificial light source.

In FIG. 7, an air conditioner including an indoor unit 5200 and an outdoor unit 5204 is an example of an electronic appliance including a power storage device 5203 according to one embodiment of the present invention. Specifically, the indoor unit 5200 includes a housing 5201, a ventilation duct 5202, the power storage device 5203, and the like. FIG. 7 illustrates the case where the power storage device 5203 is provided in the indoor unit 5200; alternatively, the power storage device 5203 may be provided in the outdoor unit 5204. Further alternatively, the power storage devices 5203 may be provided in both the indoor unit 5200 and the outdoor unit 5204. The air conditioner can receive power from the commercial power supply. Alternatively, the air conditioner can use power stored in the power storage device 5203. Specifically, in the case where the power storage devices 5203 are provided in both the indoor unit 5200 and the outdoor unit 5204, the air conditioner can be operated with the use of the power storage device 5203 according to one embodiment of the present invention as an uninterruptible power supply even when power cannot be supplied from the commercial power supply due to power failure or the like.

Note that although the separated air conditioner including the indoor unit and the outdoor unit is illustrated in FIG. 7 as an example, the power storage device according to one embodiment of the invention can be used in an air conditioner in which the functions of an indoor unit and an outdoor unit are integrated in one housing.

In FIG. 7, an electric refrigerator-freezer 5300 is an example of an electronic appliance including a power storage device 5304 according to one embodiment of the present invention. Specifically, the electric refrigerator-freezer 5300 includes a housing 5301, a door for a refrigerator 5302, a door for a freezer 5303, the power storage device 5304, and the like. The power storage device 5304 is provided in the housing 5301 in FIG. 7. Alternatively, the electric refrigerator-freezer 5300 can receive power from the commercial power supply. Alternatively, the electric refrigerator-freezer 5300 can use power stored in the power storage device 5304. Thus, the electric refrigerator-freezer 5300 can be operated with the use of the power storage device 5304 according to one embodiment of the present invention as an uninterruptible power supply even when power cannot be supplied from the commercial power supply due to power failure or the like.

Note that among the electronic appliances described above, a high-frequency heating apparatus such as a microwave and an electronic appliance such as an electric rice cooker require high power in a short time. The tripping of a circuit breaker of a commercial power supply in use of electronic appliances can be prevented by using the power storage device according to one embodiment of the present invention as an auxiliary power supply for supplying power which cannot be supplied sufficiently by a commercial power supply.

In addition, in a time period when electronic appliances are not used, specifically when the proportion of the amount of power which is actually used to the total amount of power which can be supplied by a commercial power supply source (such a proportion is referred to as usage rate of power) is low, power can be stored in the power storage device, whereby the usage rate of power can be decreased in a time period when the electronic appliances are used. For example, in the case of the electric refrigerator-freezer 5300, power can be stored in the power storage device 5304 in night time when the temperature is low and the door for a refrigerator 5302 and the door for a freezer 5303 are not often opened or closed. On the other hand, in daytime when the temperature is high and the door for a refrigerator 5302 and the door for a freezer 5303 are frequently opened and closed, the power storage device 5304 is used as an auxiliary power supply; thus, the usage rate of power in daytime can be decreased.

This embodiment can be implemented in appropriate combination with any of the above embodiments.

### Example 1

In this example, discharge characteristics and charge characteristics of a battery 1 and a comparative battery 1 were measured.

First, a method for fabricating a sample will be described.

### (Formation of Graphene Oxide Salt)

### <Oxidation of Graphite>

At the beginning, 2 g of graphite and 92 ml of concentrated sulfuric acid were mixed to prepare a mixed solution A 1. Then, 12 g of potassium permanganate was added to the mixed solution A 1 while they were stirred in an ice bath, so that a mixed solution A 2 was prepared. After the ice bath was removed and stirring was performed at room temperature for 2 hours, the resulting solution was left at 35° C. for 30 minutes so that the graphite was oxidized. Consequently, a mixed solution A 3 containing graphite oxide was formed.

### <Reduction of Metal Ion>

Next, 184 ml of pure water was added to the mixed solution A 3 while they were stirred in an ice bath, so that a mixed solution A 4 was formed. After the mixed solution A 4 was stirred in an oil bath at about 98° C. for 15 minutes so that reaction was caused, 580 ml of pure water and 36 ml of hydrogen peroxide solution (with a concentration of 30 wt %) were added to the mixed solution A 4 while they were stirred, so that unreacted potassium permanganate was deactivated. Consequently, a mixed solution A 5 containing soluble manganese sulfate and the graphite oxide was formed.

### <Collection of Graphite Oxide>

Next, after suction filtration of the mixed solution A 5 was carried out using a membrane filter with a pore size of 0.1 a precipitate A 1 was formed. Then, the precipitate A 1 and 3 wt % of hydrochloric acid were mixed, so that a mixed solution A 6 in which a manganese ion, a potassium ion, and a sulfate ion were dissolved was formed. After that, suction filtration of the mixed solution A 6 was carried out, so that a precipitate A 2 containing the graphite oxide was formed.

### <Formation of Graphene Oxide>

After the precipitate A 2 was mixed with 500 ml of pure water to obtain a mixed solution A 7, ultrasonic waves with a frequency of 40 kHz were applied to the mixed solution A 7 for an hour to separate carbon layers in the graphite oxide from each other, so that graphene oxide was formed. Note that instead of the graphene oxide, multilayer graphene oxide might be formed in some cases.

### <Collection of Graphene Oxide>

Next, centrifugation was carried out at 4000 rpm for about 30 minutes to collect a supernatant fluid containing the graphene oxide. The supernatant fluid was to be a mixed solution A 8.

### <Formation of Graphene Oxide Salt>

Next, ammonia water was added to the mixed solution A 8 so that the pH thereof was adjusted to be 11. Accordingly, a mixed solution A 9 was prepared. After that, 2500 ml of acetone was added to the mixed solution A 9 and mixing was carried out to obtain a mixed solution A 10. At this time, the graphene oxide included in the mixed solution A 8 reacted with ammonia included in the ammonia water to form graphene oxide salt as a precipitate A 3. Note that instead of the graphene oxide salt, multilayer graphene oxide salt might be formed in some cases.

### <Collection of Graphene Oxide Salt>

The precipitate A 3 was dried at room temperature in a vacuum to collect the graphene oxide salt.

### (Fabrication of Battery)

Next, a battery was fabricated. A method for fabricating a battery will be described below.

In a positive electrode, aluminum foil was used as a current collector, and a positive electrode active material layer which is a mixture of lithium iron phosphate (LiFePO₄) particles and the graphene oxide salt which was obtained through the above process was formed over the current collector.

A method for forming a lithium iron phosphate particle is shown below. Lithium carbonate (Li₂CO₃), iron oxalate (Fe₂CO₄.H₂O), and ammonium dihydrogen phosphate (NH₄H₂PO₄) were weighed out such that the molar ratio therebetween was 1:2:2, and were ground and mixed with a wet ball mill (the ball diameter was 3 mm and acetone was used as a solvent) at 400 rpm for 2 hours.

After the grinding and mixing, drying is carried out and then pre-baking was carried out at 350° C. in a nitrogen atmosphere for 10 hours, and the grinding and mixing were carried out again with the wet ball mill (the ball diameter was 3 mm) at 400 rpm for 2 hours. After that, baking was carried out at 600° C. in a nitrogen atmosphere for 10 hours.

Next, 5 wt % of graphene oxide salt and 95 wt % of lithium iron phosphate particles were mixed with NMP having a weight about triple as large as the total weight of the graphene oxide salt and the lithium iron phosphate particles, a current collector was coated with the mixture and subjected to vacuum drying at 120° C. for 60 minutes, punching was carried out to form a circular shape, and heating was carried out at 300° C. in a vacuum for 8 hours to 10 hours. Thus, a positive electrode whose active material layer has a thickness of 11 μm was formed. Note that the graphene oxide salt was used as a conductive additive and a binder.

For a negative electrode, lithium foil with a circular shape formed by punching was used.

Next, the battery 1 was fabricated using the following: a mixed solution of ethylene carbonate (EC) and diethyl carbonate (DEC) (with a volume ratio of 1:1) in which 1 mol/L of lithium hexafluorophosphate (LiPF₆) was dissolved as an electrolyte solution and polypropylene separator as a separator.

### (Measurement of Charge and Discharge Characteristics of Battery 1)

Discharge characteristics of the fabricated battery 1 were measured and then charge characteristics thereof were measured. Note that the discharge rate and the charge rate were each 0.2 C. The charging was stopped when the constant voltage reaches 4.3 V.

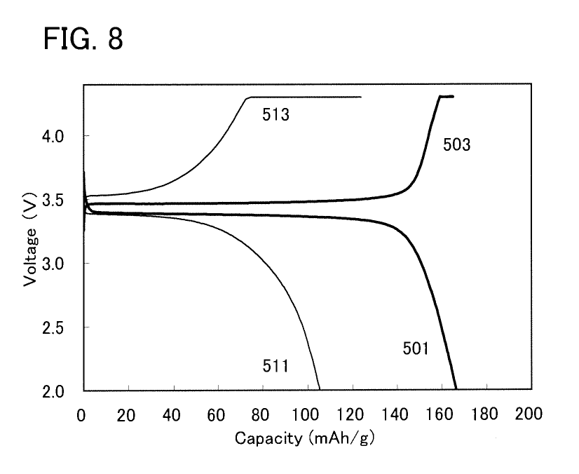

FIG. 8 shows discharge and charge characteristics of the battery. Note that the horizontal axis indicates capacity per weight of the active material of the positive electrode, and the vertical axis indicates voltage at the time of charging and discharging. A curve 501 represents discharge characteristics of the battery 1, and a curve 503 represents charge characteristics of the battery 1. FIG. 8 shows that the discharge capacity of the battery 1 is 165 mAh/g, which is close to the theoretical capacity of LiFePO₄ which is a positive electrode active material.

Next, as for a comparative battery, the charge and discharge characteristics of the comparative battery 1 including a positive electrode in which LiFePO₄ was used as the positive electrode active material, acetylene black was used as the conductive additive, and PVDF was used as the binder were measured. Note that the discharge rate was 0.2 C and the charge rate was 1 C. The charging was stopped when the constant voltage reaches 4.3 V. In addition, 85 wt % of lithium iron phosphate particles which was formed in a manner similar to that of the battery 1, 8 wt % of acetylene black, and 7 wt % of PVDF were mixed with NMP having a weight about twice as large as the total weight of the lithium iron phosphate particles, acetylene black, and the PVDF, a current collector was coated with the mixture and vacuum drying was performed at 120° C. for an hour, and pressure was applied to the mixture with a roller to increase adhesiveness between the active material and the acetylene black. After that, punching was carried out to form a circular shape, so that the positive electrode whose active material layer has a thickness of 32.4 μm was formed. The negative electrode, the electrolyte, and the separator were formed similar to those of the battery 1.

In FIG. 8, a curve 511 represents discharge characteristics of the comparative battery 1, and a curve 513 represents charge characteristics of the comparative battery 1.

From FIG. 8, it can be seen that the battery 1 has higher discharge capacity than the comparative battery 1. Accordingly, the use of graphene oxide salt for a positive electrode active material presumably allows the amount of the positive electrode active material per unit weight to be increased, so that the discharge capacity of the battery 1 can be close to the theoretical discharge capacity.

### Example 2

In this example, results of nuclear magnetic resonance (NMR) measurement of graphene oxide salt formed according to Embodiment 1 and graphene formed by a conventional method will be described.

As a sample 1, graphene oxide salt was formed in a manner similar to that of Example 1.

Further, the sample 1 was subjected to reduction treatment to form graphene (sample 2). The sample 2 was formed in a manner such that reduction treatment was carried out on the graphene oxide salt by baking the sample 1 at 300° C. in a vacuum for 10 hours.

As a comparative sample 1, graphene oxide was formed by a conventional formation method. The formation method of the comparative sample 1 will be described below.

At the beginning, 5 g of graphite and 126 ml of concentrated sulfuric acid were mixed to obtain a mixed solution A 11. Then, 12 g of potassium permanganate was added to the mixed solution A 11 while they were stirred in an ice bath, so that a mixed solution A 12 was formed. After the ice bath was removed and stirring was performed at room temperature for 2 hours, the resulting solution was left at 35° C. for 30 minutes so that the graphite was oxidized. Consequently, a mixed solution A 13 containing graphite oxide was formed.

Next, 184 ml of pure water was added to the mixed solution A 13 while they were stirred in an ice bath, so that a mixed solution A 14 was formed. After the mixed solution A 14 was stirred in an oil bath at about 95° C. for 15 minutes so that reaction was caused, 560 ml of pure water and 36 ml of hydrogen peroxide solution (with a concentration of 30 wt %) were added to the mixed solution A 14 while they were stirred, so that the potassium permanganate was deactivated. Consequently, a mixed solution A 15 containing soluble manganese sulfate and the graphite oxide was formed.

After suction filtration of the mixed solution A 15 was carried out using a membrane filter with a pore size of 1 μm, hydrochloric acid was mixed into the resulting solution and sulfuric acid was removed from the resulting solution, so that a mixed solution A 16 containing the graphite oxide was formed.

Pure water was added to the mixed solution A 16, and centrifugation was carried out at 3000 rpm for about 30 minutes to remove a supernatant fluid. Then, a process in which pure water was added to a precipitate and centrifugation was carried out to remove a supernatant fluid was repeated a plurality of times. When the pH of the mixed solution A 16 from which the supernatant fluid was removed reached about 5 to 6, ultrasonic treatment was carried out for 2 hours to separate the graphite oxide. In this manner, a mixed solution A 17 from which the graphene oxide was separated was formed.

Water in the mixed solution A 17 was removed with an evaporator, and a residue was ground in a mortar and heated in a glass tube oven at 300° C. in a vacuum for 10 hours, so that oxygen in the graphene oxide was reduced and partly released. Accordingly, graphene was formed.

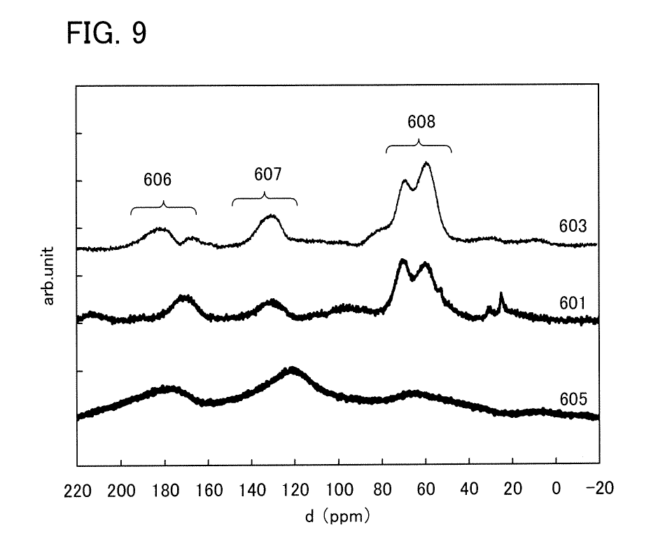

Next, ¹³C-NMR spectra of the sample 1, the sample 2, and the comparative sample 1 are shown in FIG. 9, and analysis results thereof will be described below. In FIG. 9, a curve 601 indicates a ¹³C-NMR spectrum of the sample 1, a curve 603 indicates a ¹³C-NMR spectrum of the comparative sample 1, and a curve 605 indicates a ¹³C-NMR spectrum of the sample 2.

A signal 606 represents carbonyl carbon, a signal 607 represents aromatic carbon, and a signal 608 represents aliphatic carbon. Although the signal 606 representing carbonyl carbon in the sample 1 is transferred from that in the comparative sample 1, there is no great difference between the sample 1 and the comparative sample 1 in the signal 607 representing aromatic carbon and the signal 608 representing aliphatic carbon. This shows that the graphene oxide salt obtained in Example 1 has a carbon skeleton similar to that of conventional graphene oxide.

The signal 608 representing aliphatic carbon in the sample 2 which was obtained by reducing the sample 1 was smaller than that in the comparative sample 1 and that in the sample 1, and this shows that reduction of an oxygen atom bonded to a carbon atom was enhanced.

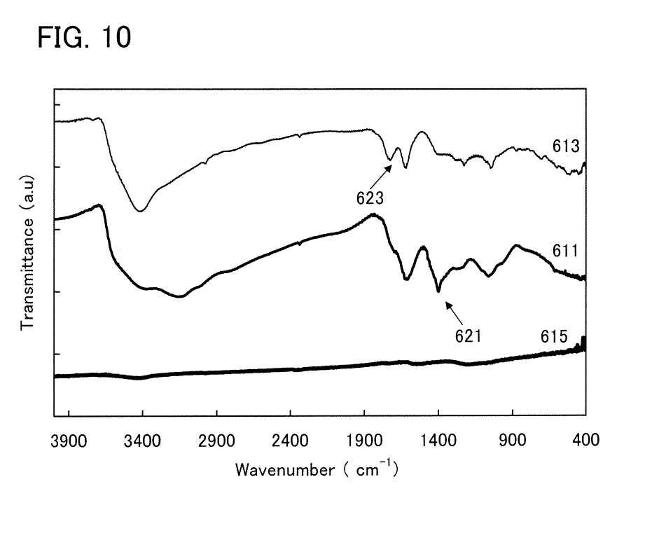

Next, FIG. 10 shows infrared absorption spectra of the sample 1, the comparative sample 1, and the sample 2, which were measured by infrared spectroscopy. In FIG. 10, whose vertical axis represents transmittances with an arbitrary unit, each curve is shown so that peak positions can be compared.

A curve 611 indicates an infrared absorption spectrum of the sample 1, a curve 613 indicates an infrared absorption spectrum of the comparative sample 1, and a curve 615 indicates an infrared absorption spectrum of the sample 2.

A peak 621 indicates an absorption peak of an ammonium group. A peak 623 indicates an absorption peak of a carboxyl group. These results suggest that the sample 1 does not contain a carboxyl group but contains an ammonium group, whereas the comparative sample 1 contains a carboxyl group. In addition, the curve 615 suggests that a carboxyl group and an ammonium group are released by carrying out reduction treatment.

From the above results, it can be seen that graphene oxide salt can be formed according to Example 1 and graphene can be formed by carrying out reduction treatment on the graphene oxide salt.

### Example 3

In this example, graphene oxide and graphene oxide salt formed according to Embodiment 1 and graphene formed by a conventional method were measured by X-ray photoelectron spectroscopy (XPS) and elemental analysis of carbon, hydrogen, and nitrogen (CHN elemental analysis).

First, a method for forming samples will be described.

### <Oxidation of Graphite>

At the beginning, 1 g of graphite and 46 ml of concentrated sulfuric acid were mixed to prepare a mixed solution A 21. Then, 6 g of potassium permanganate was added to the mixed solution A 21 while they were stirred in an ice bath, so that a mixed solution A 22 was prepared. After the ice bath was removed and stirring was performed at room temperature for 2 hours, the resulting solution was reacted at 35° C. for 30 minutes so that a mixed solution A 23 containing graphite oxide was formed.

### <Reduction of Metal Ion>

Next, 92 ml of pure water was added to the mixed solution A 23 while they were stirred in an ice bath, so that a mixed solution A 24 was formed. After the mixed solution A 24 was stirred in an oil bath at about 95° C. for 15 minutes so that reaction was caused, 280 ml of pure water and 18 ml of hydrogen peroxide solution (with a concentration of 30 wt %) were added to the mixed solution A 24 while they were stirred, so that the potassium permanganate was deactivated. Consequently, a mixed solution A 25 containing soluble manganese sulfate and the graphite oxide was formed.

### <Collection of Graphite Oxide>

Next, after suction filtration of the mixed solution A 25 was carried out using a membrane filter with a pore size of 0.1 μm, a precipitate A 21 was formed. After that, a mixed solution A 26 which was formed by adding 3% of hydrochloric acid to the precipitate A 21 and then stirring the mixture was subjected to suction filtration, so that a precipitate A 22 containing the graphite oxide was formed.

### <Formation of Graphene Oxide>

After pure water was added to the precipitate A 22 to obtain a mixed solution A 27, ultrasonic waves with a frequency of 40 kHz were applied to the mixed solution A 27 for an hour to separate carbon layers in the graphite oxide from each other, so that graphene oxide was formed. Note that instead of the graphene oxide, multilayer graphene oxide might be formed in some cases.

### <Collection of Graphene Oxide>

Next, centrifugation was carried out at 4000 rpm for about 30 minutes to collect a supernatant fluid containing the graphene oxide. The supernatant fluid was to be a mixed solution A 28.

### <Formation of Graphene Oxide Salt>

Next, ammonia water was added to the mixed solution A 28 so that the pH thereof was adjusted to be 11. Accordingly, a mixed solution A 29 was prepared. After that, acetone was added to the mixed solution A 29. At this time, the graphene oxide included in the mixed solution A 28 reacted with ammonia included in the ammonia water to form graphene oxide salt as a precipitate A 23. Note that instead of the graphene oxide salt, multilayer graphene oxide salt might be formed in some cases.

### <Collection of Graphene Oxide Salt>

The precipitate A 23 was dried at room temperature in a vacuum to collect the graphene oxide salt.

Through the above process, a sample 3 was obtained.

The sample 3 was ground in a mortar and heated in a furnace at 300° C. in a vacuum for 10 hours, so that oxygen in the graphene oxide was reduced and partly released. Accordingly, graphene was formed.

Through the above process, a sample 4 was obtained.

As a comparative sample 2, graphene oxide was formed by a conventional formation method. The formation method of the comparative sample 2 will be described below.

At the beginning, 0.25 g of graphite and 11.5 ml of concentrated sulfuric acid were mixed to prepare a mixed solution A 31. Then, 1.5 g of potassium permanganate was added to the mixed solution A 31 while they were stirred in an ice bath, so that a mixed solution A 32 was formed. After the ice bath was removed and stirring was carried out at room temperature for 2 hours, the resulting solution was reacted at 35° C. for 30 minutes so that a mixed solution A 33 containing graphite oxide was formed.

Next, 23 ml of pure water was added to the mixed solution A 33 while they were stirred in an ice bath, so that a mixed solution A 34 was formed. After the mixed solution A 34 was stirred in an oil bath at about 95° C. for 15 minutes so that reaction was caused, 70 ml of pure water and 4.5 ml of hydrogen peroxide solution (with a concentration of 30 wt %) were added to the mixed solution A 34 while they were stirred, so that the potassium permanganate was deactivated. Consequently, a mixed solution A 35 containing the graphite oxide was formed.

Next, after suction filtration of the mixed solution A 35 was carried out using a membrane filter with a pore size of 0.1 μm, a precipitate A 31 was formed. After that, a mixed solution A 36 which was formed by adding 3% of hydrochloric acid to the precipitate A 31 and then stirring the mixture was subjected to suction filtration, so that a precipitate A 32 containing the graphite oxide was formed.

After a mixed solution A 37 was formed by adding 9 ml of pure water to the precipitate A 32, centrifugation was carried out at 4000 rpm for about 30 minutes to collect a supernatant fluid containing the graphite oxide. The supernatant fluid was to be a mixed solution A 38.

Next, 10 ml of pure water was added to the mixed solution A 38, and centrifugation was carried out at 3000 rpm for about 30 minutes to remove a supernatant fluid. Then, a process in which pure water was added to the precipitate and centrifugation was carried out to remove a supernatant fluid was repeated a plurality of times. When the pH of the mixed solution A 38 from which the supernatant fluid was removed reached about 5 to 6, ultrasonic waves with a frequency of 40 kHz were applied for an hour to separate the graphite oxide. In this manner, a mixed solution A 39 in which graphene oxide was dispersed was formed.

### <Collection of Graphene Oxide>

Water in the mixed solution A 39 was removed with an evaporator, and the obtained residue was dried at room temperature in a vacuum. Accordingly, the graphene oxide was formed.

Through the above process, the comparative sample 2 was obtained.

Here, the composition ratios of carbon, oxygen, sulfur, and nitrogen which were included in the sample 3, the sample 4, and the comparative sample 2 were measured by XPS and results thereof are shown in Table 1. In the XPS of this example, Quantera SXM manufactured by PHI Inc. was used as a measurement apparatus and monochromatic AlKα ray (1.486 keV) was used for an X-ray source.

TABLE 1

| | C | O | S | N |
|---|---|---|---|---|
| | (atomic %) | (atomic %) | (atomic %) | (atomic %) |
| Sample 3 | 65.3 | 31.6 | 0.5 | 2.7 |
| Sample 4 | 86.8 | 9.9 | — | 3.2 |
| Comparative | 64.5 | 34.3 | 0.7 | 0.6 |
| Sample 2 | | | | |

Table 1 shows that the sample 3, the sample 4, and the comparative sample 2 each contain nitrogen and oxygen and that the oxygen content of the graphene oxide was able to be decreased by heat treatment carried out on the sample 3. Note that the sample 3 has lower oxygen concentration than the comparative sample 2. These results show that the oxygen content of the graphene oxide was able to be decreased according to this example.

Next, the proportions of carbon, hydrogen, nitrogen, and oxygen which were included in the sample 3 and the sample 4 (oxygen is contained only in the sample 3) were measured by CHN elemental analysis and results thereof are shown in Table 2. In the CHN elemental analysis of this example, where carbon, hydrogen, and nitrogen were measured, vario EL manufactured by Elementar Analysensysteme was used as a measurement apparatus and EMGA-920 manufactured by Horiba, Ltd. was used for measurement of oxygen. Table 1 shows the composition ratios of the elements, whereas Table 2 shows proportions of the elements. Note that the values of carbon and oxygen in Table 1 and Table 2 are different because each sample contains hydrogen.

TABLE 2

| | C | H | N | O |
|---|---|---|---|---|
| | (atomic %) | (atomic %) | (atomic %) | (atomic %) |
| Sample 3 | 43.3 | 32.8 | 2.7 | 21.3 |
| Sample 4 | 85.7 | 10.4 | 3.9 | — |

Table 2 shows that the sample 3 and the sample 4 each contain at least hydrogen and nitrogen and that the hydrogen content of the graphene oxide was able to be decreased by heat treatment carried out on the sample 3.

### Example 4

In this example, the sulfur content and the chlorine content of graphene oxide salt formed according to Embodiment 1 and graphene oxide formed by a conventional method were measured by an oxygen combustion-ion chromatography method and a flask combustion-ion chromatography method.

First, a method for forming samples will be described.

### <Oxidation of Graphite>

At the beginning, 4 g of graphite and 138 ml of concentrated sulfuric acid were mixed to prepare a mixed solution A 41. Then, 18 g of potassium permanganate was added to the mixed solution A 41 while they were stirred in an ice bath, so that a mixed solution A 42 was prepared. After the ice bath was removed and stirring was performed at room temperature for 2 hours, the resulting solution was reacted at 35° C. for 30 minutes so that a mixed solution A 43 containing graphite oxide was formed.

Next, 276 ml of pure water was added to the mixed solution A 43 while they were stirred in an ice bath, so that a mixed solution A 44 was formed. After the mixed solution A 44 was stirred in an oil bath at about 95° C. for 15 minutes so that reaction was caused, 400 ml of water and 54 ml of hydrogen peroxide solution (with a concentration of 30 wt %) were added to the mixed solution A 44 while they were stirred, so that the potassium permanganate was deactivated. Consequently, a mixed solution A 45 was formed.

Next, after suction filtration of the mixed solution A 45 was carried out using a membrane filter with a pore size of 0.45 μm, a precipitate A 41 was formed. After that, a mixed solution A 46 which was formed by adding 3% of hydrochloric acid to the precipitate A 41 and then stirring the mixture was subjected to suction filtration, so that a precipitate A 42 containing the graphite oxide was formed.

After 4000 ml of pure water was added to the precipitate A 42 to obtain a mixed solution A 47, ultrasonic waves with a frequency of 40 kHz were applied to the mixed solution A 47 for an hour to separate carbon layers in the graphite oxide from each other, so that graphene oxide was formed. Note that instead of the graphene oxide, multilayer graphene oxide might be formed in some cases.

### <Collection of Graphene Oxide>

Next, centrifugation was carried out at 9000 rpm to collect precipitated graphene oxide. Then, a process in which the same amount of pure water was added to the precipitate and centrifugation was carried out to remove a supernatant fluid was performed once, 4 times, 7 times, or 10 times in total to obtain a precipitate. These precipitates are the precipitate A 41, the precipitate A 42, a precipitate A 43, and a precipitate A 44.

Pure water was added to the above precipitate A 43, which was obtained by performing washing 4 times, and further ammonia water was added so that the pH thereof was adjusted to be 11. Accordingly, a mixed solution A 48 was prepared. After that, acetone was added to the mixed solution A 48 and mixing was carried out. At this time, the graphene oxide included in the mixed solution A 48 reacted with ammonia included in the ammonia water to form graphene oxide salt as a precipitate A 45. Note that instead of the graphene oxide salt, multilayer graphene oxide salt might be formed in some cases. After that, suction filtration was carried out on the mixed solution A 48 to obtain the precipitate A 45.

Water was removed from the precipitate A 41, the precipitate A 42, the precipitate A 43, and the precipitate A 44 with an evaporator, and each residue was ground in a mortar and the obtained powder was dried at room temperature in a vacuum. Accordingly, a comparative sample 3, a comparative sample 4, a comparative sample 5, and a comparative sample 6 were formed. Further, a sample 5 was formed when a similar process was carried out on the precipitate A 45.

Next, chlorine included in the sample 5, the comparative sample 3, the comparative sample 4, the comparative sample 5, and the comparative sample 6 was measured by an oxygen combustion-ion chromatography method. Here, each sample was combusted with QF-02 manufactured by Mitsubishi Chemical Analytech Co., Ltd. Further, sulfur contained therein was measured by a flask combustion-ion chromatography method. Here, each sample was combusted with hard glass. As an ion chromatography apparatus, DX-AQ-1120 manufactured by Dionex Corp. was used. Table 3 shows the proportions of chlorine and sulfur in each sample.

TABLE 3

| | Cl (atomic %) | S (atomic %) |
|---|---|---|
| Sample 5 | 0.03 | 0.37 |
| Comparative | 0.12 | 0.56 |
| Sample 3 | | |
| Comparative | 0.09 | 0.49 |
| Sample 4 | | |
| Comparative | 0.10 | 0.49 |
| Sample 5 | | |
| Comparative | 0.09 | 0.49 |
| Sample 6 | | |

In the comparative samples 3 to 6 in Table 3, there is a slight difference in the chlorine content. Although the sulfur content of the comparative sample 4 is lower than that of the comparative sample 3, there is no difference in the sulfur contents between the comparative sample 5 and the comparative sample 6. These results suggest that it is difficult to decrease the chlorine content and the sulfur content of graphene oxide even when the frequency of washing sulfuric acid with a mixture containing hydrochloric acid was increased, specifically even when the washing is performed more than 7 times. On the other hand, the chlorine content and the sulfur content of the sample 5 are lower than those of the comparative sample 4 are. From the above, the proportions of sulfur and chlorine included in graphene oxide can be presumably further decreased in a manner such that a basic solution and an organic solvent are mixed into a liquid containing graphene oxide, the graphene oxide and a base included in the basic solution are reacted with each other, and graphene oxide salt is formed.

### EXPLANATION OF REFERENCE

201: negative electrode current collector, 203: negative electrode active material layer, 205: negative electrode, 211: negative electrode active material, 213: graphene or multilayer graphene, 221: negative electrode active material, 221a: common portion, 221b: projected portion, 223: graphene or multilayer graphene, 307: positive electrode current collector, 309: positive electrode active material layer, 311: positive electrode, 321: positive electrode active material, 323: graphene or multilayer graphene, 400: lithium-ion secondary battery, 401: positive electrode current collector, 403: positive electrode active material layer, 405: positive electrode, 407: negative electrode current collector, 409: negative electrode active material layer, 411: negative electrode, 413: separator, 415: electrolyte, 417: external terminal, 419: external terminal, 421: gasket, 501: curve, 503: curve, 511: curve, 513: curve, 601: curve, 603: curve, 605: curve, 606: signal, 607: signal, 608: signal, 611: curve, 613: curve, 615: curve, 621: peak, 623: peak, 5000: display device, 5001: housing, 5002: display portion, 5003: speaker portion, 5004: power storage device, 5100: lighting device, 5101: housing, 5102: light source, 5103: power storage device, 5104: ceiling, 5105: wall, 5106: floor, 5107: window, 5200: indoor unit, 5201: housing, 5202: ventilation duct, 5203: power storage device, 5204: outdoor unit, 5300: electric refrigerator-freezer, 5301: housing, 5302: door for a refrigerator, 5303: door for a freezer, and 5304: power storage device.

This application is based on Japanese Patent Application serial No. 2011-179215 filed with the Japan Patent Office on Aug. 18, 2011 and Japanese Patent Application serial No. 2011-179217 filed with the Japan Patent Office on Aug. 18, 2011, the entire contents of which are hereby incorporated by reference.

1. A method for forming graphene oxide salt comprising the steps of:
forming a first precipitate by mixing graphite and an oxidizer containing an alkali metal salt in a solution;
forming a second precipitate by ionizing the oxidizer containing the alkali metal salt, which is included in the first precipitate, with an acid solution, and removing the oxidizer containing the alkali metal salt from the first precipitate;
forming a mixed solution by mixing the second precipitate and water;
separating graphene oxide from graphite oxide that is the graphite which is included in the second precipitate and oxidized by applying ultrasonic waves to the mixed solution or mechanically stirring the mixed solution, so that a dispersion liquid in which the graphene oxide is dispersed is formed; and
forming graphene oxide salt by mixing the dispersion liquid, a basic solution, and an organic solvent and reacting the graphene oxide included in the dispersion liquid and a base included in the basic solution to each other.

2. The method for forming graphene oxide salt according to claim 1, wherein the oxidizer is nitric acid and potassium chlorate; sulfuric acid and potassium permanganate; or nitric acid, sulfuric acid, and potassium chlorate.

3. The method for forming graphene oxide salt according to claim 1, wherein the acid solution is hydrochloric acid, dilute sulfuric acid, or nitric acid.

4. The method for forming graphene oxide salt according to claim 1, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

5. The method for forming graphene oxide salt according to claim 1, wherein the organic solvent is acetone, methanol, or ethanol.

6. A method for forming graphene oxide salt comprising the steps of:
forming a first mixed solution containing a first precipitate having graphite oxide and an oxidizer by mixing graphite and the oxidizer in a solution;
forming a second precipitate containing the graphite oxide by collecting the first precipitate from the first mixed solution and then removing the oxidizer from the first precipitate with an acid solution;
forming a second mixed solution in which graphene oxide is dispersed by mixing the second precipitate and water and then applying ultrasonic waves to a mixed solution or mechanically stirring the mixed solution, so that the graphene oxide is separated from the graphite oxide; and
collecting graphene oxide salt by mixing a basic solution and an organic solvent with the second mixed solution and reacting the graphene oxide included in the second mixed solution and a base to each other to precipitate the graphene oxide salt.

7. The method for forming graphene oxide salt according to claim 6, wherein the oxidizer is nitric acid and potassium chlorate; sulfuric acid and potassium permanganate; or nitric acid, sulfuric acid, and potassium chlorate.

8. The method for forming graphene oxide salt according to claim 6, wherein the acid solution is hydrochloric acid, dilute sulfuric acid, or nitric acid.

9. The method for forming graphene oxide salt according to claim 6, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

10. The method for forming graphene oxide salt according to claim 6, wherein the organic solvent is acetone, methanol, or ethanol.

11. A method for forming graphene oxide salt comprising the steps of:
forming a first precipitate by mixing graphite and an oxidizer containing an alkali metal salt in a solution;
forming a second precipitate by ionizing the oxidizer containing the alkali metal salt, which is included in the first precipitate, with an acid solution, and removing the oxidizer containing the alkali metal salt from the first precipitate;
forming a third precipitate containing graphite oxide salt by mixing the second precipitate and water, then mixing a basic solution and an organic solvent, and reacting graphite oxide that is formed by oxidizing the graphite, which is included in the second precipitate, and the basic solution; and
forming graphene oxide by mixing the third precipitate and water and separating graphene oxide salt from the graphite oxide salt included in the third precipitate.

12. The method for forming graphene oxide salt according to claim 11, wherein the oxidizer is nitric acid and potassium chlorate; sulfuric acid and potassium permanganate; or nitric acid, sulfuric acid, and potassium chlorate.

13. The method for forming graphene oxide salt according to claim 11, wherein the acid solution is hydrochloric acid, dilute sulfuric acid, or nitric acid.

14. The method for forming graphene oxide salt according to claim 11, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

15. The method for forming graphene oxide salt according to claim 11, wherein the organic solvent is acetone, methanol, or ethanol.

16. A method for forming graphene oxide salt comprising the steps of:
forming a first mixed solution containing a first precipitate having graphite oxide and an oxidizer by mixing graphite and the oxidizer in a solution;
forming a second precipitate containing the graphite oxide by collecting the first precipitate from the first mixed solution and then removing the oxidizer from the first precipitate with an acid solution;
forming a third precipitate containing graphite oxide salt by mixing the second precipitate and water, then mixing a basic solution and an organic solvent, and reacting the graphite oxide, which is included in the second precipitate, and a base; and
forming a second mixed solution in which graphene oxide salt is dispersed by mixing the third precipitate and water, and then applying ultrasonic waves to a mixed solution or mechanically stirring the mixed solution, so that the graphene oxide salt is separated from the graphite oxide salt included in the third precipitate, and collecting the graphene oxide salt included in the second mixed solution.

17. The method for forming graphene oxide salt according to claim 16, wherein the oxidizer is nitric acid and potassium chlorate; sulfuric acid and potassium permanganate; or nitric acid, sulfuric acid, and potassium chlorate.

18. The method for forming graphene oxide salt according to claim 16, wherein the acid solution is hydrochloric acid, dilute sulfuric acid, or nitric acid.

19. The method for forming graphene oxide salt according to claim 16, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

20. The method for forming graphene oxide salt according to claim 16, wherein the organic solvent is acetone, methanol, or ethanol.

21. A method for forming graphene comprising the steps of:
forming a first precipitate by mixing graphite and an oxidizer containing an alkali metal salt in a solution;
forming a second precipitate by ionizing the oxidizer containing the alkali metal salt, which is included in the first precipitate, with an acid solution, and removing the oxidizer containing the alkali metal salt from the first precipitate;
forming a mixed solution by mixing the second precipitate and water;
separating graphene oxide from graphite oxide that is the graphite which is included in the second precipitate and oxidized by applying ultrasonic waves to the mixed solution or mechanically stirring the mixed solution, so that a dispersion liquid in which the graphene oxide is dispersed is formed;
forming graphene oxide salt by mixing the dispersion liquid, a basic solution, and an organic solvent and reacting the graphene oxide included in the dispersion liquid and a base included in the basic solution to each other; and
forming graphene by reducing the graphene oxide salt.

22. The method for forming graphene according to claim 21, wherein the oxidizer is nitric acid and potassium chlorate; sulfuric acid and potassium permanganate; or nitric acid, sulfuric acid, and potassium chlorate.

23. The method for forming graphene according to claim 21, wherein the acid solution is hydrochloric acid, dilute sulfuric acid, or nitric acid.

24. The method for forming graphene according to claim 21, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

25. The method for forming graphene according to claim 21, wherein the organic solvent is acetone, methanol, or ethanol.

26. A method for forming graphene comprising the steps of:
forming a first mixed solution containing a first precipitate having graphite oxide and an oxidizer by mixing graphite and the oxidizer in a solution;
forming a second precipitate containing the graphite oxide by collecting the first precipitate from the first mixed solution and then removing the oxidizer from the first precipitate with an acid solution;
forming a second mixed solution in which graphene oxide is dispersed by mixing the second precipitate and water, and then applying ultrasonic waves to a mixed solution or mechanically stirring the mixed solution, so that the graphene oxide is separated from the graphite oxide;
collecting graphene oxide salt by mixing a basic solution and an organic solvent with the second mixed solution and reacting the graphene oxide included in the second mixed solution and a base to each other to precipitate the graphene oxide salt; and
forming graphene by reducing the graphene oxide salt.

27. The method for forming graphene according to claim 26, wherein the oxidizer is nitric acid and potassium chlorate; sulfuric acid and potassium permanganate; or nitric acid, sulfuric acid, and potassium chlorate.

28. The method for forming graphene according to claim 26, wherein the acid solution is hydrochloric acid, dilute sulfuric acid, or nitric acid.

29. The method for forming graphene according to claim 26, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

30. The method for forming graphene according to claim 26, wherein the organic solvent is acetone, methanol, or ethanol.

31. A method for forming graphene comprising the steps of:
forming a first precipitate by mixing graphite and an oxidizer containing an alkali metal salt in a solution;
forming a second precipitate by ionizing the oxidizer containing the alkali metal salt, which is included in the first precipitate, with an acid solution, and removing the oxidizer containing the alkali metal salt from the first precipitate;
forming a third precipitate containing graphite oxide salt by mixing the second precipitate and water, then mixing a basic solution and an organic solvent, and reacting graphite oxide that is formed by oxidizing the graphite, which is included in the second precipitate, and the basic solution;
forming graphene oxide by mixing the third precipitate and water and separating graphene oxide salt from the graphite oxide salt included in the third precipitate; and
forming graphene by reducing the graphene oxide salt.

32. The method for forming graphene according to claim 31, wherein the oxidizer is nitric acid and potassium chlorate; sulfuric acid and potassium permanganate; or nitric acid, sulfuric acid, and potassium chlorate.

33. The method for forming graphene according to claim 31, wherein the acid solution is hydrochloric acid, dilute sulfuric acid, or nitric acid.

34. The method for forming graphene according to claim 31, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

35. The method for forming graphene according to claim 31, wherein the organic solvent is acetone, methanol, or ethanol.

36. A method for forming graphene comprising the steps of:
forming a first mixed solution containing a first precipitate having graphite oxide and an oxidizer by mixing graphite and the oxidizer in a solution;
forming a second precipitate containing the graphite oxide by collecting the first precipitate from the first mixed solution and then removing the oxidizer from the first precipitate with an acid solution;
forming a third precipitate containing graphite oxide salt by mixing the second precipitate and water, then mixing a basic solution and an organic solvent, and reacting the graphite oxide which is included in the second precipitate and a base;
forming a second mixed solution in which graphene oxide salt is dispersed by mixing the third precipitate and water, and then applying ultrasonic waves to a mixed solution or mechanically stirring the mixed solution, so that the graphene oxide salt is separated from the graphite oxide salt included in the third precipitate, and collecting the graphene oxide salt included in the second mixed solution; and
forming graphene by reducing the graphene oxide salt.

37. The method for forming graphene according to claim 36, wherein the oxidizer is nitric acid and potassium chlorate; sulfuric acid and potassium permanganate; or nitric acid, sulfuric acid, and potassium chlorate.

38. The method for forming graphene according to claim 36, wherein the acid solution is hydrochloric acid, dilute sulfuric acid, or nitric acid.

39. The method for forming graphene according to claim 36, wherein the basic solution is an aqueous sodium hydroxide solution, an aqueous pottasium hydroxide solution, an aqueous ammonia solution, a methylamine solution, an ethanolamine solution, a dimethylamine solution, or trimethylamine solution.

40. The method for forming graphene according to claim 36, wherein the organic solvent is acetone, methanol, or ethanol.

41-42. (canceled)
